(19) United States
(12) Patent Application Publication
(10) Pub. No.: US 2018/0161412 A1
(43) Pub. Date: Jun. 14, 2018

(54) EXTENDED PROTECTION PROTEIN VACCINES AGAINST INFECTIOUS AGENTS

(71) Applicant: Cyvax, Inc., Atlanta, GA (US)

(72) Inventor: Richard Markham, Columbia, MD (US)

(73) Assignee: Cyvax, Inc., Atlanta, GA (US)

(21) Appl. No.: 15/841,935

(22) Filed: Dec. 14, 2017

Related U.S. Application Data
(60) Provisional application No. 62/434,203, filed on Dec. 14, 2016.

(51) Int. Cl. A61K 39/015; A61K 39/39; A61K 9/00; C07K 16/20; C07K 14/44

(52) U.S. Cl. CPC A61K 39/015; A61K 39/39; A61K 2039/5154; C07K 16/205; C07K 14/44; A61K 9/0019

(57) ABSTRACT

Protein-based vaccines against infectious agents, including malaria and Zika virus, are described. The protein-based vaccines include an antigen domain and an immature dendritic cell targeting domain and are administered in combination with an adjuvant.

### RELATED APPLICATIONS

This application claims the benefit under 35 U.S.C. § 119(e) of U.S. provisional application No. 62/434,203, filed Dec. 14, 2016, which is incorporated by reference herein in its entirety.

### FIELD OF THE DISCLOSURE

Provided herein, in some aspects, are protein-based vaccines against infectious agents, including malaria and Zika virus, for example. The protein-based vaccines, in some embodiments, include an antigen domain and an immature dendritic cell targeting domain and are administered in combination with an adjuvant.

### BACKGROUND OF THE DISCLOSURE

The World Health Organization estimated in 2010 that 3.3 billion people live in areas of the world that place them at risk for developing malaria. In the same year at least 219 million of these people developed the disease and 660,000 of them died, primarily infants and young children.

Malaria is caused by parasitic protozoans of the genus Plasmodium. The life cycle of Plasmodium is complex and includes several stages. First, Plasmodium sporozoites are injected into hosts (e.g., humans) by a mosquito, via the mosquito's saliva. Relatively few sporozoites, typically not more than 1-200, enter the bloodstream after a mosquito bite. Next, the sporozoites quickly leave the bloodstream and enter the host's liver cells. The sporozoites express and secrete a protein, circumsporozoite (CSP), which binds to host hepatocytes and contributes to invasion of the liver. Within the liver, the sporozoites can overcome the immune response in the absence of medical intervention. In this stage, the sporozoites divide in the liver to form merozoites in relative freedom from host defenses. The sporozoite and liver stages of infection are collectively known as the pre-erythrocytic phase. After the pre-erythrocytic stage, merozoites burst from ruptured liver cells into the blood stream, and quickly enter another protected environment, the red blood cells. Infection of red blood cells causes the agonizing symptoms of malaria, so preventing the parasite from reaching this phase is crucial for the patient's comfort and survival, as well as for reducing transmission to other humans.

Efforts to eradicate malaria have included decades of vaccine development research. Vaccines are formulations that, when introduced into the body, stimulate an immune response to protect against a disease caused by a particular pathogen, such as malaria caused by parasites of the genus Plasmodium. The immune response that vaccines elicit is specific to an antigen of the pathogen. Thus, pathogen antigens are a component of vaccines. A vaccine antigen can be an intact, but non-infectious form of a pathogen, or can be pathogen-derived protein or protein fragment.

When the immune system recognizes a vaccine antigen, it can lead to a long-term memory of the antigen so that if the antigen is encountered again, such as during infection with a live pathogen, the immune system can quickly and effectively mount a response.

When a vaccine is delivered to a subject, the antigen component of the vaccine can be taken up by antigen presenting cells of the immune system, such as immature dendritic cells (iDCs). Antigen presenting cells then migrate to lymph nodes, where they present the antigen or a fragment of the antigen to T cells. This presentation stimulates T cells that express receptors specific for the antigen to become long-term memory T cells, which will then circulate through the body in order to quickly respond if the antigen is later encountered. Antigen presentation also causes B cells that express receptors specific for the antigen to produce and secrete antibodies that will then circulate through the body to elicit a quick, robust immune response if the antigen is later encountered. Antigen-specific T cells aid in the development of the protective antibody response.

Despite decades of malaria vaccine research, these efforts have yet to generate a clinically effective vaccine. Reports from the 1960s and 70s, that immunization with irradiated sporozoites could induce sterilizing immunity to malaria in mice and humans, have provided the foundation for current sporozoite-targeted strategies in malaria vaccine development. For instance, several research projects have sought to exploit these original observations by developing a practical method for obtaining and delivering sporozoites to the billions of at risk individuals. Sporozoite vaccines can elicit immunity, however, to achieve high rates of protection, five rounds of intravenous immunization are needed (Agnandji, S. T., et al., New Engl. J. Med. 365:1863-75, 2011; Seder, R. A., et al., Science 341:1359-65, 2013). A further problem with sporozoite vaccines is that they rely on inoculation of irradiated Plasmodium sporozoites isolated from mosquitoes, a labor-intensive process not suitable for wide-spread use.

The immunodominant antigen of sporozoites is the central repeat region of the circumsporozoite protein (CSP). There is clear evidence that circumsporozoite protein (CSP)-specific antibodies, at sufficient concentration, are capable of providing sterilizing immunity against malaria infection. Recent vaccine efforts include the RTS,S/AS01 (RTS,S) vaccine, which is a recombinant protein-based vaccine engineered using regions of the CSP protein of Plasmodium falciparum as an antigen.

Although exposure to CSP antigen is known to induce a robust antibody response, problems were evident in results from clinical RTS,S vaccine trials. In the first year following completion of the vaccination protocols, vaccine efficacy was at best 50% and this level fell to 16% over the ensuing three years (Olotu, A., et al., New Engl. J. Med. 368:1111-20, 2013; Agnandji, S. T., et al., New Engl. J. Med. 365:1863-75, 2011; Rts SCTP, Lancet 2015; 386:31-45). Thus, two major problems with current efforts in malaria vaccine development include (1) the need for three or more immunizations to obtain even short term protection in settings where health care delivery can be problematic and (2) the inability of the vaccine to provide sustained protection over an extended period. Therefore, there is a need for the development of malaria vaccines that can elicit a more potent and longer-lasting immune response.

In addition to a need in the art for improved malaria vaccines, there is also a need for vaccines to combat emerging infectious agents, such as the Zika virus. Zika virus is classified as a member of the genus Flavivirus and family Flaviviridae. Dengue virus and West Nile virus are other members of this family Zika virus disease is spread to humans primarily through the bite of an infected mosquito, particularly Aedes aegypti and Aedes albopictus, but recent evidence shows that sexual transmission also occurs from infected humans.

According to the Centers for Disease Control (CDC), Zika virus was discovered in 1947 (the name refers to the Zika Forest in Uganda). Until recently, Zika infections were considered rare. However, thousands of cases have now been reported, and Zika-carrying mosquitoes are found in an increasingly large geographic area. In 2016, the Zika virus constitutes a growing public health emergency. Zika virus infection during pregnancy can cause serious birth defects including microcephaly and other severe brain defects, so prevention of infection in pregnant women, and woman intending to become pregnant, is a critical medical need. However, as of late 2016, no vaccine for Zika is available to the general public.

### SUMMARY OF THE DISCLOSURE

Provided herein, in some aspects, are protein-based malaria vaccines that elicit extended, potent immune responses with fewer than three doses. In some embodiments, the vaccines comprise (a) a fusion protein that comprises a Plasmodium circumsporozoite protein (CSP) linked to a chemokine that targets immature dendritic cells and (b) an adjuvant, wherein administration of the vaccine to subject elicits an immune response to Plasmodium CSP. The chemokine may be, for example, MIP-3a, MC148, vMIP-II (vMIP2), vMIP-I, or U83A.

In some embodiments, the adjuvant comprises a squalene-based adjuvant, while in other embodiments, the adjuvant comprises polyinosinic-polycytidylic acid (poly-IC) or a poly-IC derivative.

In some embodiments, the vaccine is formulated at a dose of 50 μg-250 μg (e.g., 50, 60, 70, 80, 90, 100, 125, 150, 175, or 200 μg).

In some embodiments, the immune response is an anti-CSP neutralizing antibody response. In some embodiments, an anti-CSP neutralizing antibody titer can be detected in the subject following administration of fewer than three doses (e.g., after a single dose or after only two doses) of the vaccine. Surprisingly, in some embodiments, protective immunity against Plasmodium infection is achieved following fewer than three doses of the vaccine.

In some embodiments, the anti-CSP neutralizing antibody titer is at least 10-fold greater than a control, wherein the control is an anti-CSP neutralizing antibody titer elicited in a subject administered a DNA vaccine comprising an adjuvant and a nucleic acid encoding a fusion protein that comprises a Plasmodium CSP linked to a chemokine that targets immature dendritic cells.

In some embodiments, a reciprocal anti-CSP neutralizing antibody titer of at least 10⁵ is detected in the subject at 6 weeks following administration of fewer than three doses (e.g., after a single dose or after only two doses) of the vaccine.

In some embodiments, following administration of the vaccine comprising a Plasmodium CSP fused to a chemokine formulated with adjuvant to a subject, the number of inflammatory cells attracted to the site of vaccine administration is at least 50% (e.g., at least 60%, 70%, 80% or 90%) greater compared to a control, wherein the control is the number of inflammatory cells attracted to the site of vaccine administration in a subject administered a vaccine comprising Plasmodium CSP fused to a chemokine formulated without adjuvant. See Table 1, herein and compare CSP alone to CSP+adjuvant, and compare MCSP alone to MCSP+adjuvant.

In some embodiments, following administration of the vaccine to a subject, the Plasmodium parasitic load is reduced by at least 90% in the subject after challenge with Plasmodium, relative to the Plasmodium parasitic load detected in an unvaccinated control subject after challenge with Plasmodium.

The Plasmodium CSP, in some embodiments, comprises a sequence at least 90% (e.g., at least 95%, at least 98%, or 100%) identical to the sequence identified by SEQ ID NO: 26.

The fusion protein, in some embodiments, comprises a sequence at least 90% (e.g., at least 95%, at least 98%, or 100%) identical to the sequence identified by any one of SEQ ID NO: 38, SEQ ID NO: 39, or SEQ ID NO: 40.

Also provided herein, in some aspects, are methods that comprise administering to a subject the protein-based vaccine of the present disclosure, wherein the vaccine elicits an immune response to Plasmodium CSP.

The vaccine may be administered intramuscularly, for example. The vaccine may be administered as a single dose, or as a prime dose and then again as a boost dose.

A subject herein is typically a human subject, and in some embodiments, is a child under the age of 5 years or an infant under the age of 1 year.

The present disclosure also provides vaccines that comprise a Zika virus antigen linked to a chemokine that targets immature dendritic cells and (b) an adjuvant, wherein administration of the vaccine to subject elicits an immune response to Zika virus antigen. Further provided herein are methods that comprise administering to a subject a Zika virus vaccine.

The protein-based vaccines, in some embodiments, include at least three components: (1) an antigen domain that elicits an immune response to a pathogen, such as a malaria parasite or Zika virus; (2) an iDC targeting domain that binds to a receptor expressed by iDCs; and (3) an adjuvant that enhances the antigen-specific immune response. The disclosed protein-based vaccines can be used as vaccinations to protect against infections such as malaria and Zika virus.

In particular embodiments, when used as a malaria vaccine, the antigen domain is the P. falciparum protein CSP or a fragment of the CSP protein. In particular embodiments, when used as a Zika virus vaccine, the antigen domain is the Zika virus E protein or Domain III of the Zika virus E protein.

In particular embodiments, the iDC targeting domain is a chemokine that binds to a chemokine receptor expressed by iDCs. In particular embodiments, the iDC targeting domain includes vMIP-I, vMIP-II, MC148, U83A, and/or MIP-3α, or a fragment thereof.

In particular embodiments, the adjuvant is a squalene-based adjuvant. In particular embodiments, the squalene-based adjuvant is MF59® (Novartis, Basel, Switzerland).

In particular embodiments, the adjuvant is alum.

In particular embodiments, the extended protection vaccine is a fusion protein with an antigen domain including a fragment of the Plasmodium CSP protein and an iDC targeting domain, delivered with an adjuvant.

In particular embodiments, the extended protection vaccine is a fusion protein with an antigen domain including all or a fragment of Domain III of the E protein of Zika virus and an iDC targeting domain, delivered with an adjuvant.

### BRIEF DESCRIPTION OF THE FIGURES

FIG. 1 shows binding of the MIP-3α-fused vaccine construct to HEK293 cells expressing CCR6. Recombinant c-Myc-tagged CSP or MCSP was incubated with either CCR6-expressing CCR6/HEK293 or parental HEK293 cells. Bound vaccine construct was detected by flow cytometry analysis of cells using PerCP/cy5.5-conjugated anti-mouse CCR6 or FITC-conjugated anti-c-Myc mAbs targeting the tag incorporated into MCCSP and CSP constructs.

FIG. 2 shows the immunogenicity of different malaria vaccine constructs. C57BL/6 mice were immunized twice with 20 μg of the different protein vaccines at three week intervals. Mice were bled three weeks after the first and second immunizations to determine specific antibody levels. The endpoint titer is reported as the highest dilution of serum at which the absorbance was twice the value obtained using pre-immunization serum. *p<0.01 comparing CSP/poly(I:C) to MCSP or CSP after the second immunization. **p<0.006 comparing MCSP/poly(I:C) to any, of the other CSP containing regimens.

FIGS. 3A and 3B show the maintenance of anti-CSP antibody concentrations after immunization as measured by ELISA. C57BL/6 mice were immunized with 20 μg MCSP+Poly(I:C) twice at a 3-week interval. Specific antibody concentrations were tested at the indicated time points. Values shown represent the absorbance at OD405 nm for simultaneously run samples (FIG. 3A) and the reciprocal of the endpoint ELISA titer (FIG. 3B) for individual mice (●) and the mean of those titers for the 5 mice in each group (---). *p=0.1**p<0.001

FIGS. 4A and 4B show malaria vaccine-mediated protection against liver stage infection at three weeks (FIG. 4A) and 23 weeks (FIG. 4B) after the final immunization of C57BL/6 mice immunized twice with 20 μg MCSP+Poly(I:C).

FIGS. 5A, 5B and 5C show the absence of antibody response to the MIP-3α component of malaria vaccine in C57BL/6 mice immunized with 20 μg hMCSP+Poly(I:C) twice at 3 week intervals. Specific antibodies against human MIP-3α (FIG. 5A), mouse MIP-3α (FIG. 5B) or PfCSP (FIG. 5C) were assayed by ELISA three weeks after the final immunization. Values shown represent absorbance at OD405 nm from serum of pre-immune or immunized mice. No reactivity against either human or marine MIP-3α significantly exceeded that of the pre-immune sera (p=1.000).

FIG. 6 shows the time course of antibody response with different adjuvants. Mice immunized twice with 20 μg MCSP and the indicated adjuvants (Poly(I:C), MF59 and Alum) at the recommended dosing were followed by ELISA for concentration of CSP-specific antibody (1:5000) dilution. P=0.36 difference in slopes; P<0.001 difference in magnitude of response.

FIG. 7 shows vaccine-mediated protection against liver stage infection 15 weeks after the final immunization. The experiment was conducted as in FIG. 4 using Addavax™ (an MF59®-like adjuvant for laboratory use) instead of poly(I:C). P<0.0001 immune vs. control mice.

FIGS. 8A and 8B show macaque antibody response to a DNA/protein combination MCSP vaccine (DNA prime boost, FIG. 8A) and a protein MCSP vaccine (FIG. 8B). FIG. 8A shows macaque antibody response to hMCSP+Vaxfectin vaccine as DNA prime boost. Macaques were immunized with 500 μg DNA and 100 μg protein formulation of vaccine, both with Vaxfectin adjuvant. ELISA was run with sera at 1:5000 dilutions. FIG. 8B shows the reciprocal titer of CSP antibody after immunization of young macaques (six or one month-old) with either 250 μg or 50 μg MCSP protein plus 250 ul MF59® (1M).

FIG. 9 shows the results of reverse transcription and amplification of RNA encoding Zika virus E protein.

FIG. 10 shows the endpoint titer for measurement of Zika virus E protein MI domain (DEIII or DE3)-specific antibodies after immunization of five C57BL/6 mice.

FIG. 11 provides a schematic depiction of the viral construct, showing antigen (Ag) and chemokine (MIP-3α), which binds to CCR6 receptor on an immature dendritic cell (DC), with antigen uptake presentation on the mature dendritic cell.

FIGS. 12A-12C show exemplary DNA and amino acid sequences for a P. falciparum malaria vaccines, including CSP and vMIP-II (FIG. 12A, SEQ ID NOs: 19 and 29); CSP and MC148 (FIG. 12B, SEQ ID NOs: 20 and 30); as well as CSP and MIP-3α (FIG. 12C, SEQ ID NOs: 10 and 31).

FIGS. 13A-13C show exemplary DNA and amino acid sequences for Zika virus vaccines, including Domain III of the Zika virus E protein and vMIP-II (FIG. 13A, SEQ ID NOs: 21 and 27); Domain III of the Zika virus E protein and MC148 (FIG. 13B, SEQ NOs: 22 and 28); as well as Domain III of the Zika virus E protein and MIP-3α (FIG. 13C, SEQ ID NOs: 23 and 32).

FIGS. 14A-14B show the long-term immunogenicity profile of a MCSP DNA) DNA vaccine in a murine model, Specific antibody against CSP concentration tested at indicated time points (FIG. 14A). Values shown represent the absorbance at OD₄₀₅ nm. Parasite-specific rRNA levels, as determined by qRT-PCT, in the livers of mice challenged with P. berghei 3 months after the final immunization (FIG. 14B). Samples were harvested 48 hours post challenge.

FIG. 15A shows a sodium dodecyl sulfate polyacrylamide gel electrophoresis (SDS-PAGE) and Coomassie stain of vMIP-II fused to Zika virus ED3 (vMIP2ED3) protein before (lane1) and after (lane2) isopropyl β-D-1-thiogalactopyranoside (IPTG) induction. FIG. 15B shows a Western-blot of vMIP2ED3 before (lane1) and after (lane2) IPTG induction using anti-ED3 monoclonal antibody (mAb). FIG. 15C shows a Western-blot of vMIP2ED3 before (lane1) and after (lane2) IPTG induction using anti-His-tag mAb.

FIG. 16 shows a SDS-PAGE and Coomassie stain of MC148 fused to Plasmodium CSP (MC148CSP) protein before lane1) and after (lane2) IPTG induction and fused to Plasmodium CSP (vMIP2CSP) protein before (lane3) and after (lane4) IPTG induction.

FIG. 17A shows an SDS-PAGE of Plasmodium CSP (CSP) protein before (lane1) and after (lane2) IPTG induction, purified CSP protein from a Ni-NTA column (lane 3), MIP-3α fused to Plasmodium CSP (MCSP) before (lane4) and after (lane 5) IPTG induction, and purified MCSP protein from a Ni-NTA column. FIG. 17B shows a Western blot of purified CSP (lane 1) and MCSP (lane 2) using an anti-CSP mAb (2A10).

FIGS. 18A and 18B show the protective ability of Macaque antibody at peak titer in C57Bl/6 mice challenged intravenously with 5×10³ transgenic P. berghei.

### DETAILED DESCRIPTION

The World Health Organization estimated in 2010 that 3.3 billion people live in areas of the world that place them at risk for developing malaria. In the same year at least 219 million of these people developed the disease and 660,000 of them died, primarily infants and young children.

Efforts to eradicate malaria have included decades of vaccine development research. Vaccines are formulations that, when introduced into the body, stimulate an immune response to protect against a disease caused by a particular pathogen, such as malaria caused by parasites of the genus Plasmodium. The immune response that vaccines elicit is specific to an antigen of the pathogen. Thus, pathogen antigens are a component of vaccines. A vaccine antigen can be an intact, but non-infectious form of a pathogen, or can be pathogen-derived protein or protein fragment.

When the immune system recognizes a vaccine antigen, it can lead to a long-term memory of the antigen so that if the antigen is encountered again, such as during infection with a live pathogen, the immune system can quickly and effectively mount a response.

When a vaccine is delivered to a subject, the antigen component of the vaccine can be taken up by antigen presenting cells of the immune system, such as immature dendritic cells (iDCs). Antigen presenting cells then migrate to lymph nodes, where they present the antigen or a fragment of the antigen to lymphocytes, such as B or T cells. This presentation stimulates T cells that express receptors specific for the antigen to become long-term memory T cells, which will then circulate through the body in order to quickly respond if the antigen is later encountered. Antigen presentation also causes B cells that express receptors specific for the antigen to produce and secrete antibodies that will then circulate through the body to elicit a quick robust immune response if the antigen is later encountered.

Despite decades of malaria vaccine research, these efforts have yet to generate a clinically effective vaccine.

In addition to a need in the art for improved malaria vaccines, there is a need for vaccines to combat emerging infectious agents, such as the Zika virus. Until recently, Zika infections were considered rare. Now, thousands of cases have been reported, and Zika-carrying mosquitoes are found in an increasingly large geographic area. In 2016 the Zika virus constitutes a growing public health emergency. Zika virus infection during pregnancy can cause serious birth defects including microcephaly and other severe brain defects, so prevention of infection in pregnant women, and woman intending to become pregnant, is a critical medical need. Currently, no vaccine for Zika is available to the general public.

The present disclosure provides protein-based vaccines that confer surprisingly long-lasting immune responses against infectious pathogens after vaccination. The protein vaccines, as provided herein, include a fusion protein that includes, in some embodiments, an antigen domain and an iDC targeting domain, such as the chemokines MIP-3α, vMIP-I, vMIP-II, U83A or MC148 (see, e.g., Dewin, D. R. et al., J. Immunology 176:544-556 (2016) for a discussion of U83A viral chemokine). The protein-based vaccines disclosed herein can be delivered with an adjuvant, such as alum, poly-IC (or a derivative thereof), or a squalene-based adjuvant. Administering the protein-based vaccine comprising an antigen fused to a chemokine formulated with an adjuvant leads to a surprisingly robust immune response, above and beyond that which would be expected from equivalent DNA-based vaccines (see, e.g., U.S. Pat. No. 8,557,248).

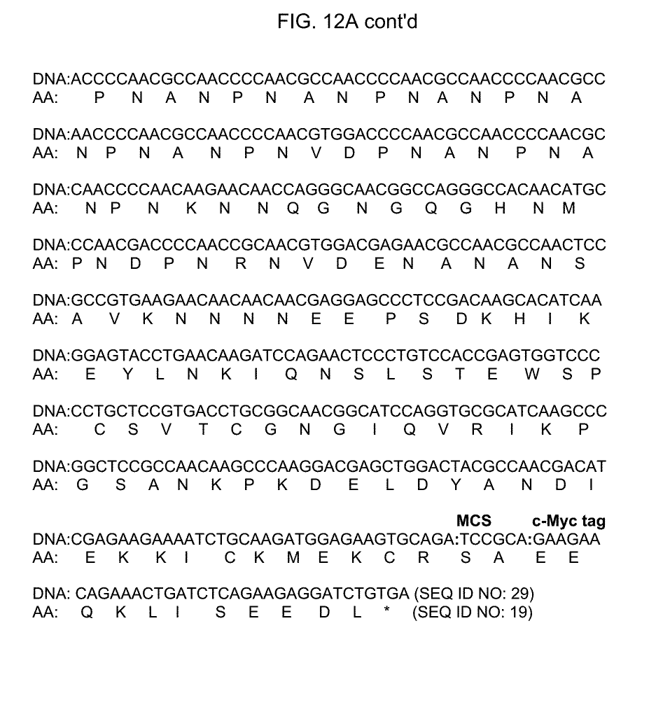

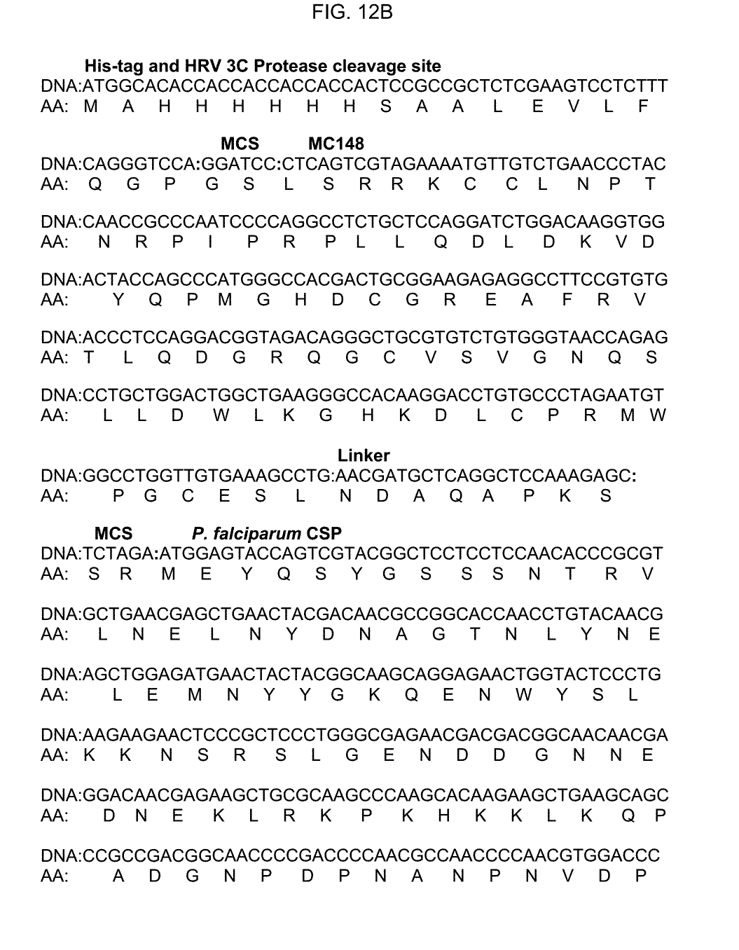

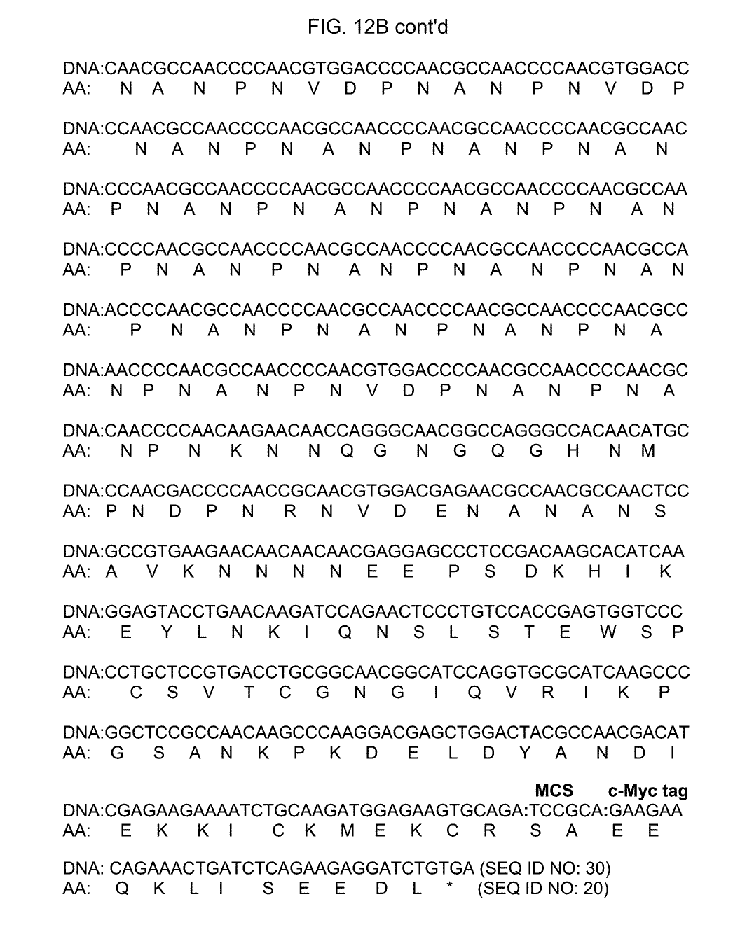

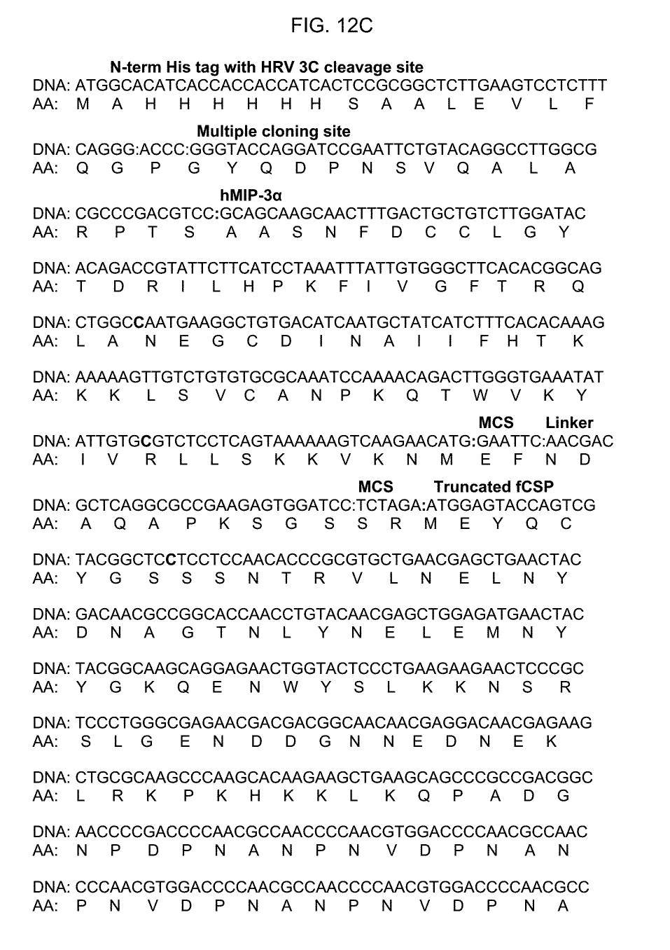

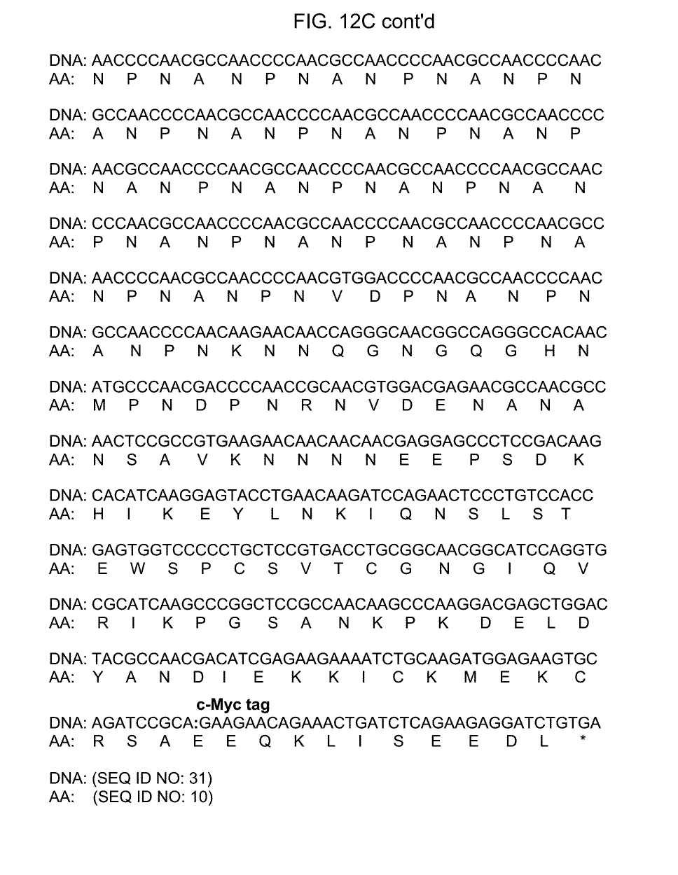

The protein-based vaccines of the present disclosure include at least three components: (1) an antigen domain that elicits an immune response to a pathogen, such as a malaria parasite or Zika virus; (2) an iDC targeting domain that binds to a receptor expressed by iDCs; and (3) an adjuvant that enhances the antigen-specific immune response. The disclosed protein-based vaccines can be used as vaccinations to protect against infections such as malaria and Zika virus. Exemplary sequences for fusion proteins that can be used for protein-based malaria vaccines, which encode a malaria antigen domain and an iDC targeting domain, include SEQ ID NOs: 38, 39, 40, 10, 19, and 20 (see FIG. 12). In some embodiments, a protein-based malaria vaccine comprises a fusion protein having a sequence that is at least 90% (e.g., at least 95% or at least 98%) identical to the sequence of SEQ ID NO: 38. In some embodiments, a protein-based malaria vaccine comprises a fusion protein having a sequence that is identical to the sequence of SEQ ID NO: 38. In some embodiments, a protein-based malaria vaccine comprises a fusion protein having a sequence that is at least 90% (e.g., at least 95% or at least 98%) identical to the sequence of SEQ ID NO: 39. In some embodiments, a protein-based malaria vaccine comprises a fusion protein having a sequence that is identical to the sequence of SEQ ID NO: 39. In some embodiments, a protein-based malaria vaccine comprises a fusion protein having a sequence that is at least 90% (e.g., at least 95% or at least 98%) identical to the sequence of SEQ ID NO: 40. In some embodiments, a protein-based malaria vaccine comprises a fusion protein having a sequence that is identical to the sequence of SEQ ID NO: 40.

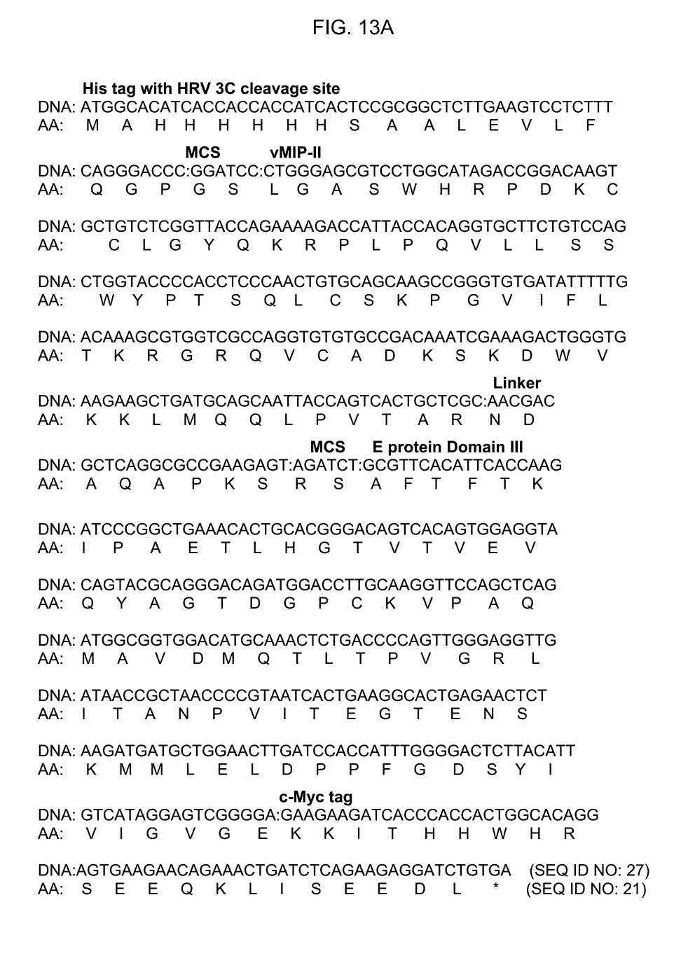

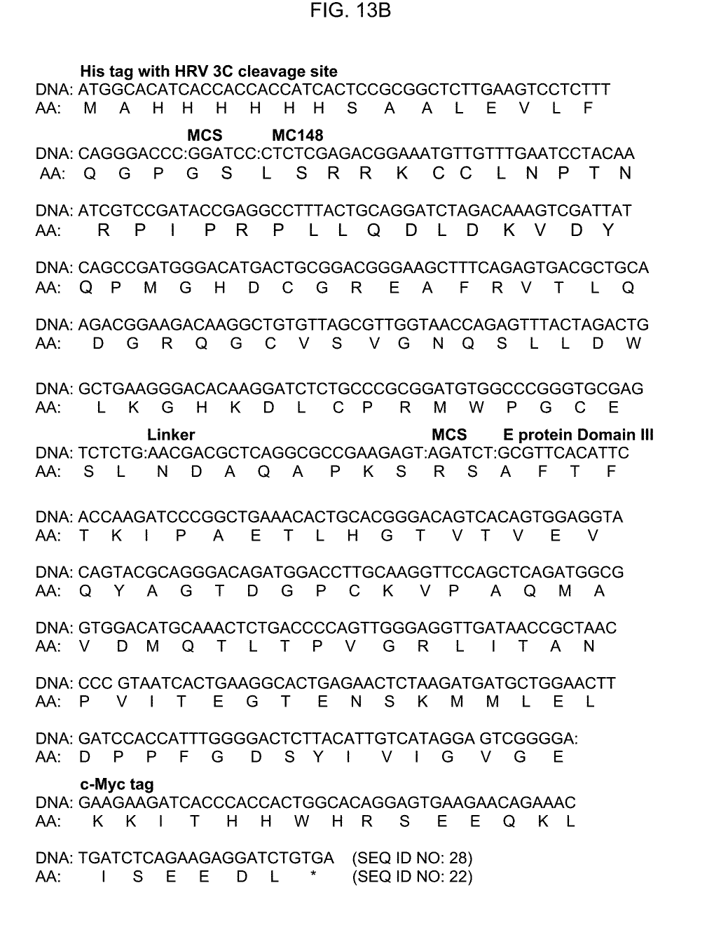

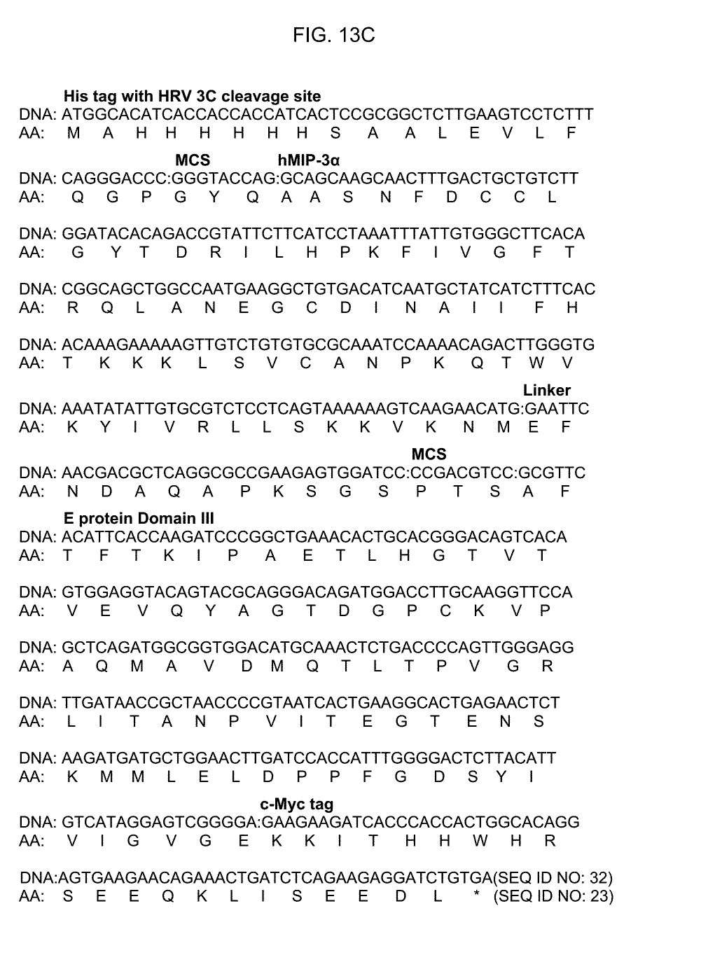

Exemplary sequences for fusion proteins that can be used for protein-based Zika vaccines, which encode a Zika antigen domain and an iDC targeting domain, include SEQ ID NOs: 41, 42, 21, 22, and 23 (see FIG. 13). In some embodiments, a protein-based Zika virus vaccine comprises a fusion protein having a sequence that is at least 90% (e.g., at least 95% or at least 98%) identical to the sequence of SEQ ID NO: 41. In some embodiments, a protein-based Zika virus vaccine comprises a fusion protein having a sequence that is identical to the sequence of SEQ ID NO: 41. In some embodiments, a protein-based Zika virus vaccine comprises a fusion protein having a sequence that is at least 90% (e.g., at least 95% or at least 98%) identical to the sequence of SEQ ID NO: 42. In some embodiments, a protein-based Zika virus vaccine comprises a fusion protein having a sequence that is identical to the sequence of SEQ ID NO: 42.

Individual components of the protein-based vaccines are now described in more detail.

Antigen Domains. The extended protection vaccines disclosed herein include an antigen domain(s) that can stimulate the body to prevent and/or fight an infection or a disease. An antigen domain is a fragment of or complete antigen that can be fused to another domain, such as a domain that enhances the immune response to the antigen domain. An antigen is a substance that, when introduced to the body stimulates an immune response, such as T cell activation or antibody production. Antigens can activate the immune system to fight infection with a pathogenic organism or virus, or can activate the immune system to clear the body of cancer cells. Vaccine antigens can include natural intact pathogens, such as a killed bacterium or virus, or a live attenuated virus. Examples of vaccine antigens that are derived from whole pathogens include the attenuated polio virus used for the OPV polio vaccine, and the killed polio virus used for the IPV polio vaccine. Vaccine antigens can include only portions, or subunits, of a pathogen, such as a single virus or bacterium protein. An example of a single protein antigen that can be used in vaccines is the L1 protein of human papillomavirus that is used for the HPV vaccine. Vaccine antigens can include proteins that are specifically or preferentially expressed by cancer cells in order to activate the immune system to fight cancer. Examples of cancer antigens include the proteins ROR1, CD19, and Mesothelin.

In particular embodiments, extended protection vaccines include a malaria antigen domain.

In particular embodiments, the antigen domain chosen is derived from a single Plasmodium protein. Examples of proteins that Plasmodium antigens can derive from include the Plasmodium proteins circumsporozoite (CSP), glutamate dehydrogenase, lactate dehydrogenase, and fructose-bisphosphate aldolase. In particular embodiments, the antigen domain used is derived from the Plasmodium circumsporozoite protein (CSP). A fragment of the Plasmodium CSP protein can also be used. An exemplary amino acid sequence of P. falciparum CSP is provided as SEQ ID NO: 26. CSP has been extensively studied in isolates from infected humans, and a variety of epitope variants are publicly known and available, including specific fragments. Any length fragment of naturally occurring or synthetic CSP, up to and including full length, is suitable for the vaccines disclosed herein, as long as it can be processed by an iDC to yield an antigen portion that will elicit a CSP-specific immune response.

In the examples herein, the circumsporozoite proteins (CSP) Plasmodium falciparum (human parasite) are used for the antigen domain. Vaccines described herein can include antigen domains derived from any malaria parasite, particularly those that infect humans. Thus, antigens from one or more (at least one) of P. falciparum, P. vivax, P. ovale, and P. malariae are suitable, particularly CSP antigens specific to each of these parasites. These four species constitute the major human Plasmodium species.

Other less common malaria parasites are also amenable to vaccine development as disclosed herein. These include the simian parasite P. knowlesi, which is documented to cause human malaria at least in Malaysia; P. simiovale, which has a circumsporozoite protein (CSP) variant of P. vivax; and P. brasilianum. Others include P. bubalis, P. juxtanucleare, P. circumflexum, P. reticulum, P. vaughani, P. minasense, P. agamae, and P. dominicum. Ongoing studies of human malaria infection may identify other species or antigen variants, and the vaccines disclosed herein can accommodate such subsequent findings without varying from the underlying mechanism.

The present disclosure also provides multivalent protein-based vaccines comprising at least two fusion proteins, wherein each fusion protein includes a different Plasmodium antigen fused to a chemokine, for example. In some embodiments, a multivalent malaria vaccine comprises (a) a fusion protein that comprises a Plasmodium CSP linked to a chemokine that targets immature dendritic cells, (b) a fusion protein that comprises Plasmodium apical membrane antigen 1 (AMA1) linked to a chemokine that targets immature dendritic cells and (c) an adjuvant, wherein administration of the vaccine to subject elicits an immune response to Plasmodium CSP. In some embodiments, a multivalent malaria vaccine comprises (a) a fusion protein that comprises a Plasmodium CSP linked to a chemokine that targets immature dendritic cells, (b) a fusion protein that comprises Plasmodium AMA1 in complex with RON2L (AMA1-RON2L) linked to a chemokine that targets immature dendritic cells and (c) an adjuvant, wherein administration of the vaccine to subject elicits an immune response to Plasmodium CSP. Other non-limiting examples of Plasmodium antigens that may be used as provided herein include Pfs25 antigen and pre-erythrocytic antigen thrombospondin-related adhesion protein (TRAP). Other Plasmodium antigens may be used, e.g., in combination with Plasmodium CSP.

In particular embodiments, extended protection vaccines include a Zika virus antigen domain. Zika virus antigen domains can include natural, intact Zika virions, or Zika virus proteins. Zika virus antigens that are derived from Zika virus proteins can include whole proteins or protein fragments. Examples of Zika virus proteins that can be used as an antigen domain include pre-membrane, envelope (E), and non-structural proteins 1-5. In particular embodiments, the Zika virus E protein can be used for the antigen domain. The E protein is responsible for virus entry into cells, and is a dominant antigen which can induce immunologic responses in infected hosts. Domain III of the E protein contains a panel of epitopes that are recognized by virus-neutralizing monoclonal antibodies. In particular embodiments, Domain III of the E protein can be used for the antigen domain (SEQ ID NO: 2).

In particular embodiments, the Zika virus strain used to derive the antigen domain can be the Fortaleza/2015 strain from Brazil. In particular embodiments, the Zika virus antigen domain can be derived from the viral strain Thailand/2014, SV0127/14. Domain III of the E protein of Zika viruses Fortaleza/2015 and Thailand/2014, SV0127/14 differ by only a single amino acid (E→D, both negatively charged). Another example of a Zika virus strain that Domain III of the E protein can be derived from is the MR-766 strain isolated in Uganda.

Immature Dendritic Cell Targeting Domains. In particular embodiments, the extended protection vaccines include an iDC targeting domain. iDCs are antigen presenting cells that initiate the adaptive immune response. The adaptive immune response is antigen-specific and leads to immune memory, whereby after the initial encounter with the antigen, immune cells that specifically bind the antigen remain on surveillance and ready to mount a quick response if the antigen is encountered again. For a vaccine to provide long-lasting protection against a pathogen or other unwanted entity, it must elicit an adaptive immune response. Lymphocytes, including B and T cells, are major cell types that are responsible for adaptive immunity. B cell immune memory is conferred by antibodies, which bind specifically to antigens. Antibodies are secreted by B cells and once B cells are activated to produce antibodies that bind a specific antigen, these antibodies can be present in the body for a very long time, up to several decades. T cell immune memory is conferred by T cell receptors, which bind specifically to peptide fragments of antigens. Once T cells are activated to respond to a specific antigen, the T cells that express T cell receptors that are specific to that antigen can become long term memory T cells, and circulate throughout the body for a very long time, up to several decades.

Prior to exposure to a specific antigen, DCs exist as immature cells (iDCs) in tissues, and possess a high capacity to phagocytize, or take up foreign particles, such as antigens. After antigen uptake, iDCs process antigens into fragments, which they display on the cell surface and this contributes to activation of the adaptive immune response. iDCs additionally receive and send immunomodulatory signals, such as through chemokines and chemokine receptors, and this can also contribute to activation of the adaptive immune response. By associating an iDC targeting domain with an antigen domain, immune activation and antigen capture by iDCs can be enhanced. A goal of the vaccine is to engage iDCs and improve the efficiency of antigen engagement by these cells, compared to use of the antigen alone. Thus, any domain that binds to iDCs can be used.

Without being bound by a specific mechanism, the data suggest that the vaccine disclosed herein works, first, by the iDC targeting domain binding to a receptor on iDCs, and then entry of the iDC targeting domain-antigen domain fusion protein into cells. Next, the antigen is processed to initiate iDC maturation and migration to the spleen, lymph nodes, and other lymphoid tissue to stimulate T cell activation and B cell production of antibodies. Fusion of an iDC targeting domain to an antigen creates a more effective interaction between iDCs and antigen, thus leading to an enhanced adaptive immune response.

In particular embodiments, the iDC targeting domain can be any protein or protein fragment that binds to a receptor expressed by iDCs. Examples of receptors expressed by iDCs include the chemokine receptors CXCR2, CXCR4, CCR1, CCR2, CCR3, CCR4, CCR5, CCR6, CCR7, and CCR8. Chemokines receptors are surface expressed proteins that bind to chemokines, a class of cytokines that can mediate chemotaxis, or cellular movement in response to a chemical stimulus. In particular embodiments, the iDC targeting domain can be any ligand that binds to a chemokine receptor expressed by iDCs.

In particular embodiments, the iDC targeting domain can be any chemokine that binds to a chemokine receptor expressed by iDCs. Examples of chemokines that bind to chemokine receptors expressed by iDCs include CXCL1, CXCL2, CXCL7(NAP2), CXCL8 (IL-8), CCL1, CCL2, CCL4, CCL5 (RANTES), CCL6, CCL7, CCL13, CCL17, CCL19, CCL20 (MIP-3α) CCL21, and CCL22.

In particular embodiments the iDC targeting domain is the chemokine macrophage inflammatory protein 3-α (MIP-3α), also referred to as Chemokine (C—C motif) ligand 20 (CCL20). MIP-3α (an exemplary full-length sequence is SEQ ID NO: 3) is used in the Examples of this disclosure and is a chemokine that binds to the receptor CCR6, which is expressed on the surface of iDCs.

In particular embodiments, the iDC targeting domain is CXCL2. CXCL2 binds to the CXCR2 expressed on the surface of iDCs and is also known as GRO2; GROβ; MIP2; MIP2α; SCYB2; MGSA-β; MIP-2α; or CINC-2α.

Suitable chemokines also include those disclosed in U.S. Patent Publication 2015/0359869.

In particular embodiments, the iDC targeting domain is a molecule other than a chemokine that is capable of binding a chemokine receptor expressed by iDCs. For example, the CCR6 receptor also interacts with human β-defensins, as demonstrated by the ability of β-defensin to displace a chemokine ligand for CCR6 (Yang, D. et al. Science 286: 525-528, 1999). Suitable β-defensins include natural, recombinant, and synthetic forms of human β-defensin 1 and human β-defensin 2, which possess chemotactic activity towards iDCs. Portions of these β-defensins that retain the ability to bind to an iDC receptor, including CCR6, are also suitable for use in the disclosed extended protection vaccines.

In particular embodiments, a fragment or portion of a β-defensin protein can be used as the iDC targeting domain. Other suitable defensins include a-defensins, which also bind to receptors expressed by iDCs.

In the context of the disclosed vaccine, any protein that can specifically bind to CCR6, or compete with a CCR6 ligand, particularly MIP-3α or β-defensin 1 or 2, is suitable to achieve the goal of enhancing the iDC response to antigen. CCR6 is a chemokine receptor expressed on the surface of iDCs and memory T cells.

In particular embodiments, any protein that can specifically bind to CCR8 or compete with a CCR8 ligand, such as CCL1 or CCL8, is suitable as an iDC targeting domain. CCR8 is a chemokine receptor expressed by various immune cells, such as iDCs.

In particular embodiments, a viral protein that binds to a chemokine receptor expressed by iDCs can be used. Examples of viral proteins that bind to chemokine receptors expressed by iDCs include vMIP-I, vMIP-II, MC148, and U83A.

In particular embodiments, the viral chemokine vMIP-II can be used. vMIP-II is encoded by Kaposi's sarcoma-associated herpesvirus, and has been reported to bind with high affinity to a number of CC and CXC chemokine receptors. vMIP-II binds to CCR1, CCR5, CCR8 and CCR2 receptors (Kledal, T. N. et al., Science 277:1656-1659, 1997). vMIP-II can enhance the production of immunity to lymphoma-derived antigens in a manner dependent upon chemokine receptors on antigen-presenting cells (Biragyn, A. et al. Blood 104: 1961-9, 2004). An exemplary polypeptide sequence of vMIP-II mature form of the protein is: LGASWHRPDKCCLGYQKRPLPQVLLSSWYPTSQLCSKPGVIFLTKRG RQVCADKSKDWVKKLMQQLPVTA (SEQ ID NO: 4) (recombinant viral MIP2 protein ab201382, Abcam, Cambridge Mass., USA). Exemplary vMIP-II as encoded by mRNA, including the signal peptide, is: MDTKGILLVAVLTALLCLQSGDTLGASWHR PDKCCLGYQKRPLPQVLLSSWYPTSQLCSKPGVIFLTKRGRQVCADKSKDWVKKLMQ QLPVTAR (SEQ ID NO: 5).

In particular embodiments, the iDC targeting domain can be vMIP-I or a fragment thereof. vMIP-I is discussed for example in Dairaghi, D. J., et al., J Biol Chem 1999; 274:21569-74. vMIP-I is a C—C type chemokine encoded by the Kaposi's sarcoma herpesvirus HHV-8. vMIP-I binds to and induces cytosolic [Ca(2+)] signals in human T cells selectively through CCR8, a CC chemokine receptor associated with Th2 lymphocytes.

According to Lüttichau, H. R. et al. J Exp Med. 2000; 3; 191(1):171-80., the MC148 CC chemokine was obtained from the human poxvirus molluscum contagiosum (MCV). In competition binding using radiolabeled endogenous chemokines as well as radiolabeled MC148, MC148 bound with high affinity only to CCR8. MC148 is shown in SEQ ID NO: 6

In particular embodiments, U83A can be used as the iDC targeting domain. U83A is a protein expressed by leukotropic human herpesvirus (HHV-6), which binds to chemokine receptors including CCR1, CCR4, CCR6, CCR8, and CCR5.

Linkers. In particular embodiments, the iDC targeting domain and the antigen domain are linked together as a fusion protein by including a linker between the two domains. Linkers, or spacers, are used to separate domains of proteins in order to preserve the bioactivity of each domain, and maintain proper folding/stability of the protein.

In particular embodiments, a flexible linker is used. A flexible linker is a series of amino acids between two domains that can bend and rotate. Flexible linkers often include small amino acids such as glycine, serine or threonine. Examples of flexible linkers include a series of glycine molecules, or a series of glycine molecules followed by a serine (known as a Gly-Ser linker).

In particular embodiments, the iDC targeting domain-antigen domain fusion protein can include the linker sequence NDAQAPKSGS (SEQ ID NO: 33) or NDAQAPKS (SEQ ID NO: 7). For more information regarding use of this linker to fuse a chemokine to an antigen domain, see Biragyn A, et al. Nat Biotechnol. 1999. 17:253-258.

Methods to Express the Antigen Domains and/or iDC Targeting Domains. In particular embodiments, the DNA that encodes for the protein vaccine can be introduced into an expression vector, such as a plasmid. Multiple cloning sites, which contain DNA sequences that are recognized by restriction enzymes, can facilitate the insertion of the protein vaccine DNA into the vector. In particular embodiments, DNA constructs (such as expression vectors) that encode the proteins of interest can be introduced into cells to induce protein expression and the cells can be harvested to extract the protein of interest. The DNA encoding the protein of interest can be included in an expression vector that also contains sequences that control gene expression, such as promoter sequences. 5′ and 3′ untranslated regions can be encoded upstream and downstream of the protein coding sequence in order to enhance expression. For example, a 5′ untranslated leader sequence and a 3′ polyadenylation sequence can be used. In particular embodiments, the DNA can be introduced into cells for protein expression by heat-shock transformation. In particular embodiments, DNA can be introduced into cells for protein expression by transfection, electroporation, impalefection or hydrodynamic delivery. In particular embodiments, the DNA used for protein expression can be delivered in the form of a viral vector. In particular embodiments, the protein of interest can be harvested from lysed cells, and purified. Protein purification can be performed using size-exclusion chromatography, or by a chromatography technique that isolates the protein based on a protein-tag, such as a 6× histidine tag or a c-myc tag. The histidine tag can be encoded adjacent to a sequence recognized and cleaved by a protease, to facilitate removal of the histidine tag after protein purification. An example of a protease that can be used to remove a histidine tag from a protein is the human rhinovirus 3C protease. An exemplary sequence for an N-terminal histidine tag followed by a human rhinovirus 3C recognition sequence is: MAHHHHHHSAALEVLFQGP (SEQ ID NO: 8). An exemplary sequence for a c-myc tag is: SAEEQKLISEEDL (SEQ ID NO: 9).

Adjuvants. Two components of the vaccine, the antigen and the iDC targeting domain, are discussed above. To further achieve an effective vaccine according to this disclosure, materials and methods are employed to enhance availability of the vaccine for iDC processing. One such method employs an adjuvant.

The term “adjuvant” refers to material that enhances the immune response to an antigen and is used herein in the customary use of the term. The precise mode of action is not understood for all adjuvants, but such lack of understanding does not prevent their clinical use for a wide variety of vaccines, whether protein-based or DNA-based. Traditionally, some adjuvants physically trap antigen at the site of injection, enhancing antigen presence at the site and slowing its release. This in turn prolongs and/or increases the recruitment and activation of APCs, such as in this case iDCs.

In particular embodiments a squalene-based adjuvant is used. Squalene is part of the group of molecules known as triterpenes, which are all hydrocarbons with 30 carbon molecules. Squalene can be derived from certain plant sources, such as rice bran, wheat germ, amaranth seeds, and olives, as well as from animal sources, such as shark liver oil. In particular embodiments, the squalene-based adjuvant is MF59®, which is an oil-in-water emulsion (Novartis, Basel, Switzerland; see Giudice, G D et al. Clin Vaccine Immunol. 2006 September; 13(9):1010-3). An example of a squalene-based adjuvant that is similar to MF59® but is designed for preclinical research use is Addavax™ (InvivoGen, San Diego, Calif.). MF59 has been FDA approved for use in an influenza vaccine, and studies indicate that it is safe for use during pregnancy (Tsai T, et al. Vaccine. 2010. 17:28(7):1877-80; Heikkinen T, et al. Am J Obstet Gynecol. 2012. 207(3):177). In particular embodiments, squalene-based adjuvants can include 0.1%-20% (v/v) squalene oil. In particular embodiments, squalene-based adjuvants can include 5% (v/v) squalene oil. In particular embodiments, the squalene-based adjuvant is AS03, which includes α-tocopherol, squalene, and polysorbate 80 in an oil-in-water emulsion (GlaxoSmithKline; see Garcon N et al. Expert Rev Vaccines. 2012 March; 11(3):349-66).

In particular embodiments, polyinosinic:polycytidilyic acid (also referred to as poly(I:C) is used. Poly(I:C) is a synthetic analog of double-stranded RNA that stimulates the immune system. In particular embodiments, Poly-ICLC (Hiltinol) is used (Ammi R et al. Pharmacol Ther. 2015 February; 146:120-31). In particular embodiments, Poliu-IC12U (Ampligen) is used (Martins K A et al. Expert Rev Vaccines. 2015 March; 14(3):447-59).

In particular embodiments the adjuvant alum can be used. Alum refers to a family of salts that contain two sulfate groups, a monovalent cation, and a trivalent metal, such as aluminum or chromium. Alum is an FDA approved adjuvant. In particular embodiments, vaccines can include alum in the amounts of 1-1000 ug/dose or 0.1 mg-10 mg/dose.

In particular embodiments, the adjuvant Vaxfectin® (Vical, Inc., San Diego, Calif.) can be used. Vaxfectin® is a cationic lipid based adjuvant that can be used for DNA or protein vaccines.

Compositions for Administration. Vaccines of the disclosure can be formulated into pharmaceutical compositions for administration including a vaccine of the disclosure can be formulated in a variety of forms, e.g., as a liquid, gel, lyophilized, or as a compressed solid. The particular form will depend upon the particular indication being treated and will be apparent to one of ordinary skill in the art.

An example of a pharmaceutical composition is a solution designed for parenteral administration. Although in many cases pharmaceutical solution formulations are provided in liquid form, appropriate for immediate use, such parenteral formulations can also be provided in frozen or in lyophilized form. In the former case, the composition must be thawed prior to use. The latter form is often used to enhance the stability of the active compound contained in the composition under a wider variety of storage conditions, as it is recognized by those or ordinary skill in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

Parenterals can be prepared for storage as lyophilized formulations or aqueous solutions by mixing, as appropriate, the composition having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from 2 mM to 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris.

Preservatives can be added to retard microbial growth, and are typically added in amounts of 0.2%-1% (w/v). Suitable exemplary preservatives for use with the present disclosure include phenol, benzyl alcohol, meta-cresol, methyl paraben, propyl paraben, octadecyldimethylbenzyl ammonium chloride, benzalkonium halides (e.g., benzalkonium chloride, bromide or iodide), hexamethonium chloride, alkyl parabens such as methyl or propyl paraben, catechol, resorcinol, cyclohexanol and 3-pentanol.

Isotonicifiers can be added to ensure isotonicity of liquid compositions and include polyhydric sugar alcohols, trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the vaccine or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, and glycerol; polyethylene glycol; amino acid polymers; sulfur-containing reducing agents, such as urea, glutathione, thioctic acid, sodium thioglycolate, thioglycerol, alpha-monothioglycerol and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the vaccine composition. Additional miscellaneous excipients include bulking agents or fillers (e.g., starch), chelating agents (e.g., EDTA), antioxidants (e.g., ascorbic acid, methionine, vitamin E) and cosolvents.

The vaccine composition can also be entrapped in microcapsules prepared, for example, by coascervation techniques or by interfacial polymerization, for example hydroxymethylcellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences.

Parenteral formulations to be used for in vivo administration generally are sterile. This is readily accomplished, for example, by filtration through sterile filtration membranes.

Suitable examples of sustained-release vaccine compositions include semi-permeable matrices of solid hydrophobic polymers containing the composition, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the PROLEASE® (Alkermes, Inc., Waltham, Mass.) technology or LUPRON DEPOT® (injectable microspheres composed of lactic acid-glycolic acid copolymer and leuprolide acetate; Abbott Endocrine, Inc., Abbott Park, Ill.), and poly-D-(−)-3-hydroxybutyric acid. While polymers such as ethylene-vinyl acetate and lactic acid-glycolic acid enable release of molecules for long periods, such as up to or over 100 days, certain hydrogels release compounds for shorter time periods.

The administration of the vaccines can be performed in a variety of ways, including orally, subcutaneously, intravenously, intracerebrally, intranasally, transdermally, intraperitoneally, intramuscularly, intrapulmonary, intrathecally, vaginally, rectally, intraocularly, or in any other acceptable manner. The formulations can be administered continuously by infusion, although bolus injection is acceptable, using techniques well known in the art, such as pumps (e.g., subcutaneous osmotic pumps) or implantation. In some instances, the vaccine compositions can be directly applied as a solution or spray.

Subjects include individuals in need of prophylactic and/or therapeutic treatment. In particular embodiments a subject can be a human subject. In particular embodiments, the subject can be a research animal subject (such as a mouse, a rat, or a monkey), a veterinary animal subject (such as a dog, a cat, or an animal found in a zoo), or a livestock subject (such as a horse, a pig, a chicken, a turkey, or a cow). Much research on vaccines and adjuvants involves use of animal models. For example, mice can be used for malaria research by employing transgenic P. berghei engineered to express CSP from P. falciparum. The transgenic P. berghei infect mice, yet they express antigen from the strain of Plasmodium that infects humans. This approach enables testing of vaccines targeting P. falciparum CSP in mice. As another example, owl monkeys, or Aotus monkeys, can be used for malaria research. Owl monkeys are naturally resistant to malaria parasites and are a useful model for researching malaria immunity. Immunization protocols in these monkeys are described in US. Patent Publication US2015/0359869.

In some embodiments, the subject is a child under the age of 5 years (e.g., 4, 3, 2 years or 1 year). In some embodiments, the subject is an infant under the age of 1 year (e.g., 9 months, 6 months, or 3 months). In some embodiments, no more than two doses of the vaccine of the present disclosure is administered to a child or infant, while in other embodiments, more than two doses may be required to elicit protective immunity. Thus, in some embodiments, the vaccine may be administered (to an infant, child or adult (e.g., elderly or immunocompromised adult)) as one, two, three or more doses.

In particular embodiments, compositions disclosed herein can be administered for therapeutic purposes. A therapeutically effective amount of the vaccine can include an effective amount, a prophylactic treatment, and a therapeutic treatment. A therapeutic vaccine is a vaccine that is intended to treat an existing condition, by enhancing the immune systems response against a pathogen or another disease-causing target.

In particular embodiments, the vaccines disclosed herein can be administered to subjects as prophylactic treatment, or treatment for a disease that has not yet been acquired. For example, the extended protection vaccine that includes a malaria antigen can be administered to subjects either living in or travelling through regions that are high-risk for malaria acquisition. In particular embodiments, compositions including a Zika virus antigen can be administered to subjects either living in or travelling through regions that are high-risk for Zika virus acquisition.

For administration of a therapeutically effective amount of a composition disclosed herein, initial estimates can be made based on in vitro and in vivo assays, as well as animal model experiments. Exemplary doses can include 0.01 μg/kg, 0.02 μg/kg, 0.03 μg/kg, 0.04 μg/kg, 0.05 μg/kg, 0.06 μg/kg, 0.07 μg/kg, 0.08 μg/kg, 0.09 μg/kg, 0.1 μg/kg, 0.2 μg/kg, 0.3 μg/kg, 0.4 μg/kg, 0.5 μg/kg, 0.6 μg/kg, 0.7 μg/kg, 0.8 μg/kg, 0.9 μg/kg, 1 μg/kg, 2 μg/kg, 3 μg/kg, 4 μg/kg, 5 μg/kg, 6 μg/kg, 7 μg/kg, 8 μg/kg, 9 μg/kg, 10 μg/kg, 20 μg/kg, 30 μg/kg, 40 μg/kg, 50 μg/kg, 60 μg/kg, 70 μg/kg, 80 μg/kg, 90 μg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 20 mg/kg, 30 mg/kg, 40 mg/kg, 50 mg/kg, 60 mg/kg, 70 mg/kg, 80 mg/kg, 90 mg/kg, and 100 mg/kg. The vaccines can be administered in a single dose, or in multiple doses. Additional administrations of vaccine doses are known as boosters. Boosters can be delivered 1, 2, 3, 4, 5, 6, or more times per week, year or decade, but this can be increased or decreased depending on factors determined by a physician, such as the subject's condition, or a subject's current immune status. In some embodiments, fewer than three doses are administered to a subject. In some embodiments, two doses of the vaccine are administered to a subject. In some embodiments, three doses of the vaccine are administered to a subject.

Effects of use. In particular embodiments, the therapeutic effectiveness of a composition can be demonstrated through induction of an immune response in a subject against an antigen presented by the vaccine. An “immune response” can be defined as activation of the innate and/or adaptive immune responses. An innate immune response is a non-specific response to an antigen and happens quickly after an antigen is encountered. When an antigen is sensed, the innate response can lead to an immune response attack on the antigen, which may or may not be sufficient to clear the infection. The innate immune response also helps activate the adaptive immune response.

The extended protection vaccine can be deemed effective if it induces a robust adaptive immune response against the antigen delivered by the vaccine. To determine if a robust adaptive immune response is elicited by the vaccine, circulating antibodies that are specific to the antigen of interest can be measured. An antibody titer is the quantification of the presence of specific antibodies present in a sample taken from a subject. An example of a technique used to measure antibody titer is the enzyme-linked immunosorbent assay (ELISA). An antibody titer can be measured to determine if the extended protection vaccine elicits an antibody response. For example, a CSP-specific antibody titer can be performed to determine if an extended protection vaccine that includes a CSP antigen elicits an antibody response. A vaccine can be determined to effectively elicit an adaptive immune response if the vaccine antigen specific antibody titer of a subject who receives the vaccine is higher than the vaccine antigen specific antibody titers for subjects who have not been exposed to the antigen, and the difference between these results is statistically significant.

In particular embodiments, the extended protection vaccines can decrease a subject's risk of acquiring malaria and/or decrease the severity of malaria infection. Malaria can be diagnosed microscopically by examining a drop of a patient's blood using a microscope. Symptoms of malaria include fever, chills, nausea, and vomiting. Evidence that a vaccine is protective against malaria can include negative diagnostic malaria test results and a lack of malaria symptoms. A decreased severity of malaria infection can be evident by a reduction in symptoms, such as a lower fever or decreased vomiting and/or nausea.

In particular embodiments, the extended protection vaccines can decrease a subject's risk of acquiring Zika and/or decrease the severity of Zika infection. Acquisition of Zika virus can be confirmed by diagnostic test that can detect the presence of Zika virus nucleic acids in a patient's blood sample. Zika infection symptoms include fever, rash, joint pain, and red eyes. Evidence that a vaccine is protective against Zika can include negative diagnostic Zika test results and a lack of Zika symptoms. A decrease in the severity of infection can cause a reduction in symptoms, such as lack of fever, or a less severe fever, and a reduction in severity of pain symptoms.

### Exemplary Embodiments

1. A vaccine comprising (i) a protein comprising (a) an antigen domain; and (b) at least one immature dendritic cell targeting domain, and (ii) an adjuvant.
2. A vaccine of embodiment 1, wherein said antigen domain and said immature dendritic cell targeting domain are joined by a linker.
3. A vaccine of embodiment 2 wherein said linker comprises SEQ ID NO: 7 or SEQ ID NO: 33.
4. A vaccine of embodiment 1 wherein said adjuvant comprises a squalene-based adjuvant.
5. A vaccine of embodiment 4 wherein said squalene-based adjuvant comprises MF59.
6. A vaccine of embodiment 1 wherein said adjuvant is alum.
7. A vaccine of embodiment 1 wherein said immature dendritic cell targeting domain binds to CCR6 and/or CCR8 on the surface of immature dendritic cells.
8. A vaccine of embodiment 1 wherein said immature dendritic cell targeting domain comprises a chemokine.
9. A vaccine of embodiment 8 wherein said chemokine comprises at least one of MIP-3α, vMIP-I, vMIP-II, U83A and MC148.
10. A vaccine of embodiment 1 wherein said immature dendritic targeting domain comprises human β-defensin.
11. A vaccine of embodiment 1 wherein said antigen domain is derived from a Plasmodium protein.
12. A vaccine of embodiment 11 wherein said Plasmodium comprises a species that infects humans.
13. A vaccine of embodiment 12 wherein said Plasmodium species that infects humans is selected from the group consisting of P. falciparum, P. vivax, P. ovale, and P. malariae.
14. A vaccine of embodiment 11 wherein said Plasmodium protein comprises circumsporozoite (CSP).
15. A vaccine of embodiment 14 wherein the CSP comprises SEQ ID NO: 26.
16. A vaccine of embodiment 1 wherein said antigen domain is derived from a Zika virus protein.
17. A vaccine of embodiment 16 wherein said Zika virus protein comprises E protein or a fragment thereof.
18. A vaccine of embodiment 17 wherein said fragment of E protein comprises SEQ ID NO: 2.
19. A method of reducing liver-stage malaria infection in a human at risk of malaria infection, said method comprising administering to said human a protein vaccine of embodiment 1.
20. A method of embodiment 19 wherein an antibody titer is measured and antibodies to said antigen domain are detectable within the blood of said human for at least 23 weeks after administering said vaccine.
21. A method of embodiment 19 wherein an antibody titer to said vaccine antigen in the blood is measured after vaccination to determine the need for additional vaccine administration.
22. A method of embodiment 19 wherein said vaccine is administered more than once.
23. A method of embodiment 19 wherein said vaccine is administered to said human by injection.
24. A method of embodiment 23 wherein said injection is intramuscular.
25. A method of providing or increasing immunity to Zika virus infection in a human at risk of Zika infection, said method comprising administering to said human a protein vaccine of embodiment 1.
26. A method of embodiment 25 wherein an antibody titer is measured and antibodies to said antigen domain are detectable within the blood of said human for at least 23 weeks after administering said vaccine.
27. A method of embodiment 25 wherein an antibody titer to said vaccine antigen in the blood is measured after vaccination to determine the need for additional vaccine administration.
28. A method of embodiment 25 wherein said vaccine is administered more than once.
29. A method of embodiment 25 wherein said vaccine is administered to said human by injection.
30. A method of embodiment 29 wherein said injection is intramuscular.
31. A method of enhancing efficacy of a vaccine which includes (i) an antigen domain and (ii) an immature dendritic cell targeting domain, said method comprising adding to said vaccine an adjuvant.
32. A method of embodiment 31 wherein said adjuvant is a squalene-based adjuvant.
33. A method of embodiment 31 wherein said adjuvant is alum.
34. A method of embodiment 31 wherein said antigen domain is derived from a Zika virus protein.
35. A method of embodiment 31 wherein said antigen domain is derived from a Plasmodium protein.

### Exemplary Sequences

(SEQ ID NO: 1)
Linker: EFNDAQAPKSGSSR
(SEQ ID NO: 24)
Linker: NDAQAPKRSTCiTS
(SEQ ID NO: 25)
Linker: NDAQAPKRSTKL

It should be understood that the linker used in any one of the constructs below (underlined) may be replaced with a different linker or may be omitted from the construct.

Plasmodium CSP
(SEQ ID NO: 26)
MEYQCYGSSSNTRVLNELNYDNAGTNLYNELEMNYYGKQENWYSLKKNSR
SLGENDDGNNEDNEKLRKPKHKKLKQPADGNPDPNANPNVDPNANPNVDP
NANPNVDPNANPNANPNANPNANPNANPNANPNANPNANPNANPNANPNA
NPNANPNANPNANPNANPNANPNANPNVDPNANPNANPNKNNQGNGQGHN
MPNDPNRNVDENANANSAVKNNNNEEPSDKHIKEYLNKIQNSLSTEWSPC
SVTCGNGIQVRIKPGSANKPKDELDYANDIEKKICKMEKCR
MIP-3α
(SEQ ID NO: 34)
AASNFDCCLGYTDRILHPKFIVGFTRQLANEGCDINAIIFHTKKKLSVCA
NPKQTWVKYIVRLLSKKVKNMEF
vMIP2
(SEQ ID NO: 35)
GASWHRPDKCCLGYQKRPLPQVLLSSWYPTSQLCSKPGVIFLTKRGRQVC
ADKSKDWVKKLMQQLPVTAR
MC148
(SEQ ID NO: 36)
LSRRKCCLNPTNRPIPRPLLQDLDKVDYQPMGHDCGREAFRVTLQDGRQG
CVSVGNQSLLDWLKGHKDLCPRMWPGCESL
Zika Virus EDIII
(SEQ ID NO: 37)
VSYSLETAAFTFTKIPAETLHGTVTVEVQYAGTDGPCKVPAQMAVDMQTL
TPVGRLITANPVITEGTENSKMMLELDPPFGDSYIVIGVGEKKITHHWHR
S
MIP-3α-CSP
(SEQ ID NO: 38)
AASNFDCCLGYTDRILHPKFIVGFTRQLANEGCDINAIIFHTIKKKLSVC
ANPKQTWVKYIVRLLSKKVKNMEFNDAQAPKSGSSRMEYQCYGSSSNTRV
LNELNYDNAGTNLYNELEMNYYGKQENWYSLKKNSRSLGENDDGNNEDNE
KLRKPKHKKLKQPADGNPDPNANPNVDPNANPNVDPNANPNVDPNANPNA
NPNANPNANPNANPNANPNANPNANPNANPNANPNANPNANPNANPNANP
NANPNANPNANPNVDPNANPNANPNKNNQGNGQGHNMPNDPNRNVDENAN
ANSAVKNNNNEEPSDKHIKEYLNKIQNSLSTEWSPCSVTCGNGIQVRIKP
GSANKPKDELDYANDIEKKICKMEKCR
vMIP2-CSP
(SEQ ID NO: 39)
GASWHRPDKCCLGYQKRPLPQVLLSSWYPTSQLCSKPGVIFLTKRGRQVC
ADKSKDWVKKLMQQLPVTARNDAQAPKRSTGTSMEYQCYGSSSNTRVLNE
LNYDNAGTNLYNELEMNYYGKQENWYSLKKNSRSLGENDDGNNEDNEKLR
KPKHKKLKQPADGNPDPNANPNVDPNANPNVDPNANPNVDPNANPNANPN
ANPNANPNANPNANPNANPNANPNANPNANPNANPNANPNANPNANPNAN
PNANPNANPNVDPNANPNANPNKNNQGNGQGHNMPNDPNRNVDENANANS
AVKNNNNEEPSDKHIKEYLNKIQNSLSTEWSPCSVTCGNGIQVRIKPGSA
NKPKDELDYANDIEKKICKMEKCR
MC148CSP
(SEQ ID NO: 40)
LSRRKCCLNPTNRPIPRPLLQDLDKVDYQPMGHDCGREAFRVTLQDGRQG
CVSVGNQSLLDWLKGHKDLCPRMWPGCESLNDAQAPKRSTGTSMEYQCYG
SSSNTRVLNELNYDNAGTNLYNELEMNYYGKQENWYSLKKNSRSLGENDD
GNNEDNEKLRKPKHKKLKQPADGNPDPNANPNVDPNANPNVDPNANPNVD
PNANPNANPNANPNANPNANPNANPNANPNANPNANPNANPNANPNANPN
ANPNANPNANPNANPNANPNVDPNANPNANPNKNNQGNGQGHNMPNDPNR
NVDENANANSAVKNNNNEEPSDKHIKEYLNKIQNSLSTEWSPCSVTCGNG
IQVRIKPGSANKPKDELDYANDIEKKICKMEKCR
vMIP2-EDIII
(SEQ ID NO: 41)
GASWHRPDKCCLGYQKRPLPQVLLSSWYPTSQLCSKPGVIFLTKRGRQVC
ADKSKDWVKKLMQQLPVTARNDAQAPKRSTKLVSYSLCTAAFTFTKIPAE
TLHGTVTVEVQYAGTDGPCKVPAQMAVDMQTLTPVGRLITANPVITEGTE
NSKMMLELDPPFGDSYIVIGVGEKKITHHWHRS
MC148-EDIII
(SEQ ID NO: 42)
LSRRKCCLNPTNRPIPRPLLQDLDKVDYQPMGHDCGREAFRVTLQDGRQG
CVSVGNQSLLDWLKGHKDLCPRMWPGCESLNDAQAPKRSTKLVSYSLCTA
AFTFTKIPAETLHGTVTVEVQYAGTDGPCKVPAQMAVDMQTLTPVGRLIT
ANPVITEGTENSKMMLELDPPFGDSYIVIGVGEKKITHHWHRS

The Examples below are included to demonstrate particular embodiments of the disclosure. It should be appreciated by those of ordinary skill in the art that the techniques disclosed in the Examples represent techniques and compositions discovered by the inventor to function well in the practice of the disclosure, and thus can be considered to constitute preferred modes for its practice. However, those of ordinary skill in the art should, in light of the present disclosure, appreciate that many changes can be made in the specific particular embodiments which are disclosed and still obtain a like or similar result without departing from the spirit and scope of the disclosure.

### Examples

In the following examples, differences among groups were analyzed by an ANOVA test (Stata Corp). Value of p<0.05 was considered to be significant. For malaria experiments, where indicated, P. berghei parasites were used for challenge. Sporozoites were obtained by hand dissection of infected mosquito salivary glands. The isolated sporozoites were suspended in HBSS medium containing 1% normal mouse serum.

All challenges were accomplished by injecting 5×10³ sporozoites in the tail vein. Humoral immune responses to the immunodominant B cell epitope was measured using variants of CSP-specific ELISA assays. Abbreviations are used as follows: M refers to MIP-3α; CSP refers to a segment of the Plasmodium circumsporozoite protein (from P. falciparum, as indicated in the specific methods); and MCSP refers to a fusion protein of MIP-3α and CSP.

### Example 1. Protein Production and Purification; Mouse Immunization

The codon-optimized P. falciparum CSP (3D7) DNA containing 22 of the central NANP repeat coding region sequences and with deletions of the N-terminal 20 aa signaling sequence and the C-terminal 22 aa anchor region fused via a spacer encoding NDAQAPKSGS (SEQ ID NO: 33) with the gene encoding human MIP-3α was used to express MIP-3α-CSP chimeric protein (MCSP, SEQ ID NO: 10). CSP and MCSP sequences were cloned into pET-47b (Novagen Inc., Madison, Wis.) and proteins were expressed in BL21 DE3 competent cells (New England Biolabs, Ipswich, Mass.).

Protein purification was undertaken using nickel-affinity chromatography (Qiagen, Valencia, Calif., USA) and endotoxin was removed by two-phase extraction with Triton X-114 (Persson, C., et al., J. Immunol. 169:6681-5, 2002).

Protein concentration was measured by Bradford assay (BioRad, Hercules, Calif.). Endotoxin concentration was determined using the ToxinSensor™ Chromogenic LAL Endotoxin Assay Kit (Genscript, N.J.). All protein used for immunization had final endotoxin levels below 10 EU/ml. Purity of proteins used for immunization and ELISA was confirmed by sodium dodecyl sulfate (SDS)-polyacrylamide gel electrophoresis (PAGE) for visualization with Coomassie Brilliant Blue staining and Western-blot analysis undertaken using the anti-CSP antibody (2A10) (Luo, K., et al., PloS one 9:e90413, 2014).

Murine CCR6-expressing HEK293 cells (HEK293CCR6) were used in Flow cytometric analysis methods. Trypsinized HEK293CCR6 or HEK293 Cells (2×10⁶ cells/ml) were incubated with 20 μg purified CSP or MCSP for 30 min on ice in PBS with 2% BSA. The interaction between MCSP and CCR6 was estimated by two-color flow cytometry analysis of cells, using PerCP/cy5.5-conjugated anti-mouse CCR6 (BioLegend, San Diego, Calif.) or FITC-conjugated anti-c-Myc mAbs (Abcam, Inc. Cambridge, Mass.).

Six- to eight-week-old female C57BL/6 (H-2b) mice were obtained from Charles River Laboratory (Charles River Laboratories, Inc., Wilmington, Mass.) and maintained in a pathogen-free micro-isolation facility in accordance with the National Institutes of Health guidelines for the humane use of laboratory animals. For immunization, C57BL/6 mice were immunized with 20 μg purified CSP or MCSP in PBS with or without the Toll-Like Receptor 3-targeting adjuvant polyinosinic-polycytidilic acid, (Poly (I:C)) (Invivogen, San Diego, Calif.). For Poly (I:C) formulation, 50 μg of Poly(I:C) was mixed with the vaccine protein. The mixture was administered intramuscularly in both anterior tibialis muscles in a total volume of 50 μl per leg.

Mice were immunized at weeks 0 and 3. Transgenic P. berghei sporozoites expressing a truncated P. falciparum CSP (Persson, C., et al., J. Immunol. 169:6681-5, 2002) were used for challenge. Sporozoites were obtained by hand dissection of salivary glands of Anopheles stephensi mosquitoes maintained in the Johns Hopkins Malaria Research Institute insectary. The isolated sporozoites were suspended in HBSS medium containing 1% normal mouse serum. Challenges to evaluate vaccine effect on hepatic parasite load were accomplished by injecting 5×10³ sporozoites by tail vein.

Humoral immune responses to the immunodominant B cell epitope of the P. falciparum CSP protein were measured using a previously described method (Foquet, L., et al., J. Clin. Invest. 124:140-4, 2014).

Briefly, ELISA plates were coated with 2 μg of recombinant PfCSP overnight at room temperature (RT). Plates were blocked with 2% BSA for 30 min at RT and then serially diluted serum samples were added and incubated for 2 h. After washing six times, peroxidase labeled goat anti-mouse IgG (Santa Cruz Biotechnology Inc., Dallas, Tex.) was added at a dilution of 1:4,000 and incubated at RT for 1 h. After washing six times, ABTS Peroxidase substrate (KPL, Gaithersburg, Md.) was added for development and incubated for 1 h. The data were collected using the Synergy HT (BioTek Instruments, Inc, Winooski, Vt.). The endpoint ELISA titer is reported as the highest dilution of serum at which the average absorbance was twice the value obtained using pre-immunization serum. For the ELISA testing anti-MIP-3α antibody, recombinant mMIP-3α and hMIP-3α were employed (BioLegend, Inc., San Diego, Calif.).

### Example 2. Real-Time PCR for Liver Stage Sporozoites

Quantitative real-time PCR was used for the detection and quantification of the liver stage of Plasmodium sporozoites. Two pairs of specific primers were designed to amplify the parasite 18s rRNA sequence. The forward primer was 5′-TGGGAGATTGGTTTTGACGTTTATGT-3′ (SEQ ID NO: 11) and the reverse primer was 5′-AAGCATTAAATAAAGCGAATACATCCTTAC-3′ (SEQ ID NO: 12). Values were normalized against measurements of mouse actin mRNA in the same samples. The primers used for mouse actin were as follows: 5′-GTCCCTCACCCTCCCAAAAG-3′ (SEQ ID NO: 13) (forward) and 5′-GCTGCCTCAACACCTCAACCC-3′ (SEQ ID NO: 14) (reverse). The reactions were performed in a final volume of 20 μl using SYBR green PCR Master Mix (2×) from Applied Biosystems and processed with ABI StepOne Real-time PCR system (Applied Biosystems).

Differences in infiltrating CD11c⁺ cells, sporozoite rRNA, and antibody concentrations among groups were analyzed by an ANOVA test (Stata Corp, College Station, Tex.). A value of p<0.05 was considered to be significant.

### Example 3. E. coli-Derived MCSP Binds to CCR6 In Vitro

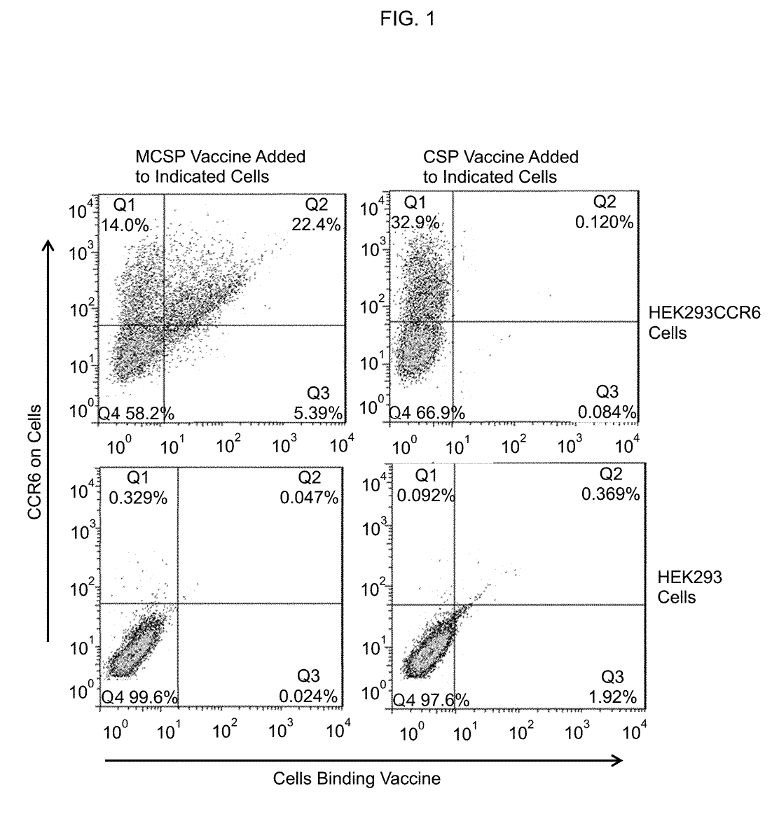

Expression and purification of the cloned and purified human MIP-3α-CSP fusion protein and the CSP protein were confirmed by ELISA and Western blot analysis using anti-CSP mAb (2A10). Both human and murine MIP-3α bind to murine CCR6 (Varona, R., et al., FEBS Lett. 440:188-94, 1998). The functional integrity of MCSP was tested by flow cytometric analysis of binding of the MIP-3α bearing CSP construct vs. the standard CSP vaccine construct lacking the fused chemokine component. FIG. 1 shows binding of the MIP-3α-fused vaccine construct to HEK293 cells expressing CCR6. Recombinant c-Myc-tagged CSP or MCSP was incubated on ice with either CCR6-expressing CCR6/HEK293 or parental HEK293 cells for 30 min.

Bound vaccine construct was detected by two-color flow cytometry analysis of cells using PerCP/cy5.5-conjugated anti-mouse CCR6 or FITC-conjugated anti-c-Myc mAbs (BioLegend, San Diego, Calif.) targeting the tag incorporated into MCSP and CSP constructs. As shown in FIG. 1, only the MIP-3α-fused CSP vaccine construct bound to the CCR6-expressing cells and the construct did not bind to the parental HEK293 cells.

### Example 4. The MIP-3α-Fused Vaccine Construct Enhances Cell Accumulation at the Site of Immunization

The impact of the presence or absence of the MIP-3α fusion component on the cellular infiltrate that accumulates at the immunization site was examined. Mice were immunized with PBS or 20 μg of the different vaccine proteins with or without Poly(I:C). After 48 hours, the anterior tibialis muscle was harvested from euthanized mice and the infiltrating cells were extracted. Total cells were counted followed by staining of the cells with APC-labeled CD11c antibody. Table 1 shows that the combination of the adjuvant Poly(I:C) and MCSP is the most potent attractant of cells to the site of inoculation compared to Poly(I:C), CSP, CSP combined with Poly (I:C), and a control group receiving only PBS (P<0.001).

TABLE 1
Impact of Different Immunization Regimens on
Cellular Infiltrate at Site of Immunization

| | Total cell | Total CD11c+ cell | % CD11c+ |
|---|---|---|---|
| | number (×10⁻⁴) | number (×10⁻⁴) | cells |
| Group | (mean ± SEM) | (mean ± SEM) | (mean ± SEM) |
| PBS (control) | 8.16 ± 0.64 | 0.56 ± 0.12 | 6.73 ± 1.17 |
| Poly(I:C) | 10.36 ± 1.84 | 1.07 ± 0.16 | 10.63 ± 1.37 |
| CSP | 13.90 ± 0.53 | 1.34 ± 0.15 | 9.63 ± 0.91 |
| CSP/Poly(I:C) | 13.66 ± 0.76 | 1.35 ± 0.24 | 9.73 ± 1.27 |
| MCSP | 31.11 ± 4.67^{a} | 2.64 ± 0.45 ^{a} | 8.53 ± 0.81 |
| MCSP/Poly(I:C) | 51.36 ± 8.92 ^{b,c} | 3.97 ± 0.39 ^{b,c} | 8.30 ± 1.76 |

^{a} p < 0.05 MCSP vs. PBS, Poly(I:C), CSP, CSP/Poly(I:C) groups.
^{b} p ≤ 0.001 MCSP/Poly(I:C) vs. PBS, Poly(I:C), CSP, CSP/Poly(I:C) groups.
^{c} p > 0.05 < 0.1 MCSP/Poly(I:C) vs. MCSP

The enhancement was also observed when MCSP was compared to Poly(I:C), CSP, CSP combined with Poly (I:C), or a control group receiving PBS (P<0.05). While adding Poly(I:C) to CSP did not enhance the cellular infiltrate, its addition to the MCSP vaccine construct resulted in marked enhancement of the cellular infiltrate (P=0.078). In all groups the proportion of infiltrate contributed by CD11c+ cells was equivalent. The above results were surprising. Previously, Luo et al. (PLoS One. 2014 Mar. 5; 9(3):e90413) examined the impact of the presence or absence of MIP3α in an DNA vaccine construct encoding MIP-3α fused to CSP on the cellular infiltrate that accumulated at the site of immunization. Table 1 of Luo et al. shows that the recruitment of cells, and specifically dendritic cells, was not enhanced by inoculating with adjuvant and the fusion MIP3α-CSP DNA construct compared to adjuvant and CSP DNA without the MIP3α component. Thus, the immunological enhancement observed with the combination of adjuvant with the MIP-3α component of the DNA vaccine cannot be attributed to the chemoattractant functions of the chemokine. With the protein vaccine of the present disclosure, however, the combination of chemokine and adjuvant resulted in marked enhancement of the cellular infiltrate at the site of vaccination. Thus, administering the protein vaccine that includes an adjuvant leads to a surprisingly robust antibody response with extended (long-lasting) protection capabilities, above and beyond that which would be expected from administering an equivalent DNA vaccine.

### Example 5. Comparison of Antibody Responses Elicited by the Different Vaccine Constructs

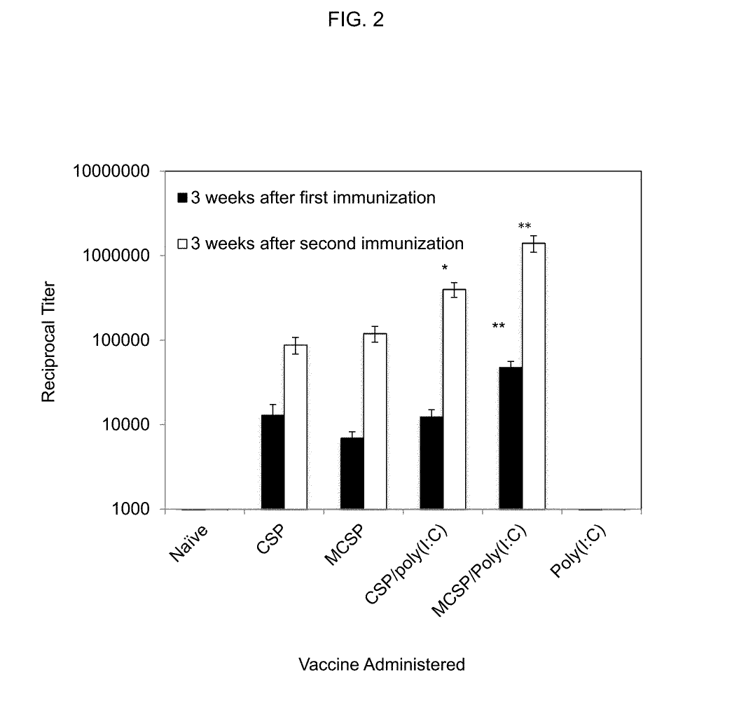

C57BL/6 mice were immunized twice with 20 μg of the different protein vaccines at a three-week interval. Naïve mice and mice injected with Poly (I:C) alone served as negative controls. Mice were bled 3 weeks after the first and second immunizations to determine specific antibody levels. CSP-specific humoral immune responses were measured by ELISA. The differences in endpoint dilution titers are shown in FIG. 2. The results indicate that fusion of MIP-3α to the CSP or CSP alone resulted in similar antibody responses. After the second immunization, the response to CSP plus Poly(I:C) was enhanced compared to the response to CSP alone or to MCSP (p<0.01). However, after two immunizations the response to the combination of Poly(I:C) plus the MIP-3α containing CSP vaccine was significantly greater than the responses to the other CSP-containing vaccine regimens (p<0.006). Reciprocal titers exceeded 10⁶.

### Example 6. Persistence of the Antibody Response to the Combination of MCSP and Poly(I:C)

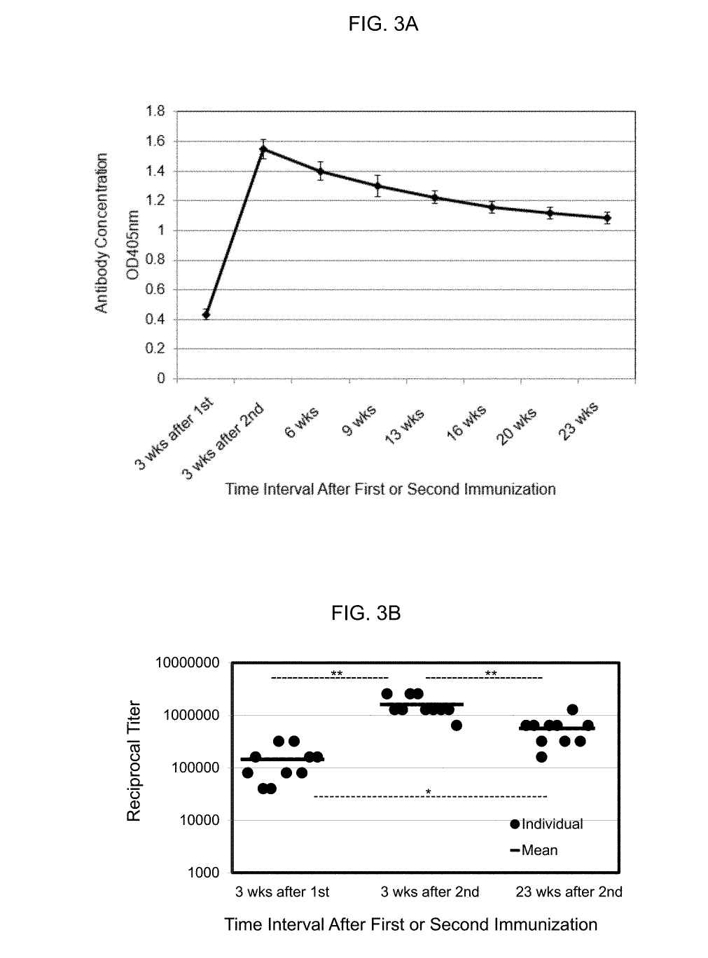

To examine the persistence of the humoral immune response induced by MCSP plus Poly(I:C) vaccination, mice were vaccinated two times (21 days apart) with 20 μg MCSP antigen and 50 μg of Poly(I:C). Serum samples were collected once every three or four weeks for up to 23 weeks after the second immunization. CSP-specific immune responses were measured by ELISA at the different time points. The OD₄₀₅ nm-time curve (FIG. 3A) shows that the antibody level slowly declined during this period.

At twenty-three weeks, the end point titer (FIG. 3B) was one third of the peak titer observed 3 weeks after the second immunization. However, because the reciprocal antibody titer was so high at three weeks after the second immunization (1.6×10⁶), the reciprocal titer at 23 weeks post the second immunization was still elevated to 6.4×10⁵.

### Example 7. Protective Efficacy of Combination of MCSP and Poly(I:C) Regimen Following Sporozoite Challenge

To evaluate in a clinically relevant manner the biological significance of the decline in reciprocal antibody titers at 23 weeks post the second immunization, the immunized mice were challenged three or 23 weeks after the final immunization with 5×10³ transgenic P. berghei sporozoites obtained by dissection of the salivary glands of infected A. stephensi mosquitoes. This challenge is one order of magnitude greater than the maximum sporozoite challenge typically encountered by humans following the bite of an infected mosquito. Similarly challenged immunologically naïve mice served as controls. Liver sections recovered 48 hours post-challenge from five or six mice from each of the immunization groups were assayed by qRT-PCR for the level of Pf 18s rRNA present.

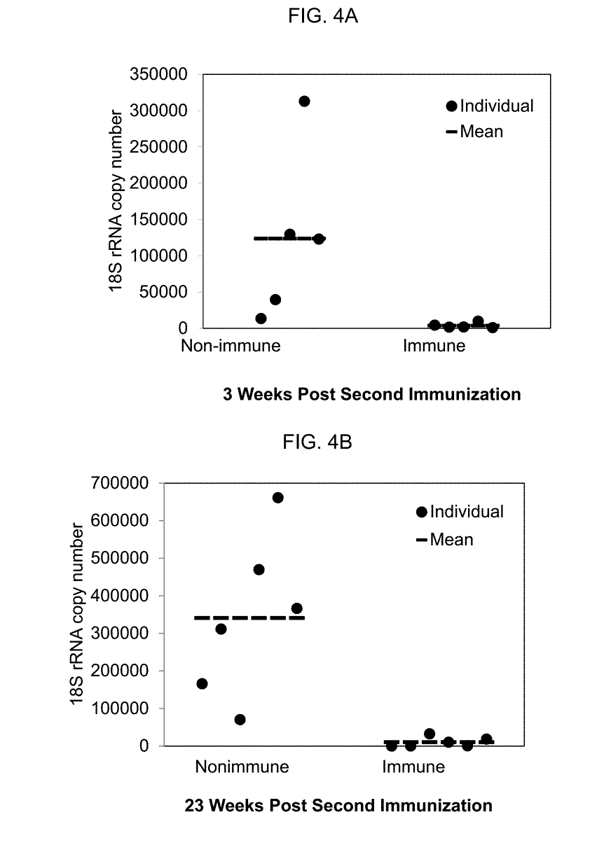

FIG. 4 shows malaria vaccine-mediated protection against liver stage infection three and 23 weeks after the final immunization. C57BL/6 mice were immunized twice with 20 μg MCSP+Poly(I:C). Three weeks (FIG. 4A) or twenty-three weeks (FIG. 4B) after the final immunization, mice were challenged with 5×10³ transgenic P. berghei sporozoites expressing P. falciparum CSP. Antibody levels (a) in pre-challenge sera were detected by ELISA and parasite-specific rRNA levels (b) in the liver were determined by quantitative RT-PCR on samples obtained 48 hours post challenge. All quantitative RT-PCR results were normalized against the expression of actin. Results in (b) indicate the sporozoite copy numbers from the livers of individual mice (●) and the mean in each group (---).

Significantly, the results showed that compared to unimmunized mice, mice immunized with MCSP plus Poly(I:C) had a 97% reduction in the transgenic P. berghei rRNA load in the liver with challenges occurring either 3 weeks (P=0.003) or 23 weeks after the second immunization (P=0.05). These results were unexpected in view of data obtained from similar experiments using a DNA-based vaccine encoding CSP fused to MIP3α formulated with adjuvant, administered as three doses, as described below (see Example 8).

### Example 8. Comparative Studies on Long-Term Immunogenicity of the DNA MCSP Vaccine and Protection

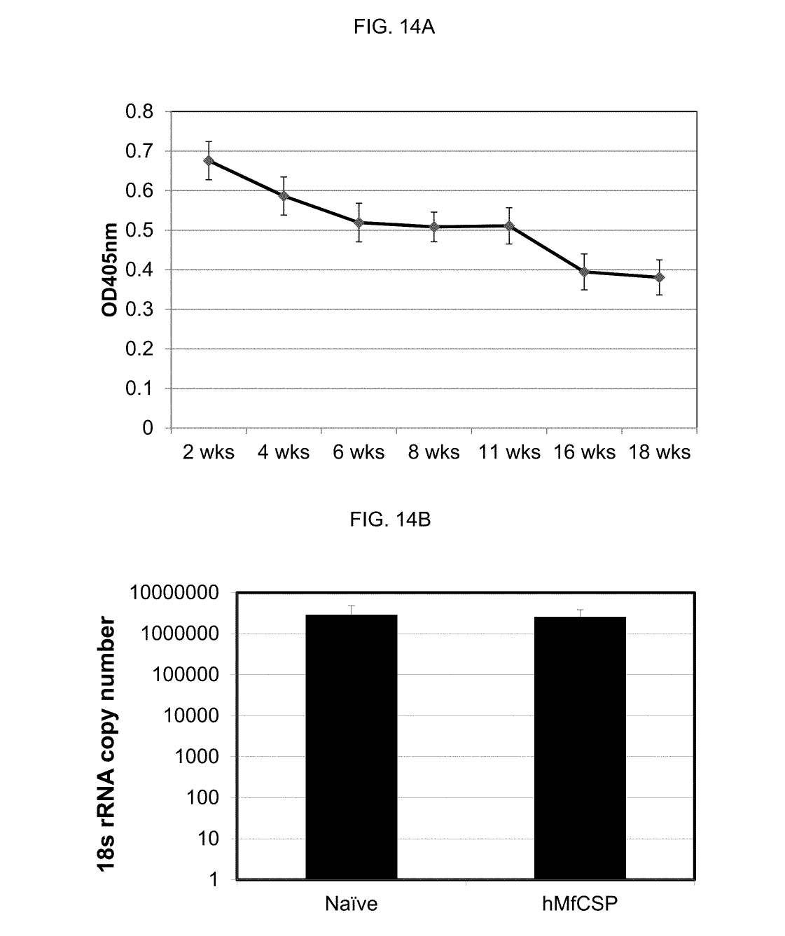

C57BL/6 mice were immunized with 5 μg of the MIP-3α fused Pf CSP and with 5 μg of Vaxfectin 3 times at 2-week intervals. Specific antibody against Pf CSP concentrations was tested from 2-18 weeks post-immunization (FIG. 14A). 3 months after the final immunization, mice were challenged with 5×10³ transgenic P. berghei sporozoites expressing P. falciparum CSP (FIG. 14B). Parasite-specific rRNA levels in the liver were determined by quantitative RT-PCR on samples obtained 48 hours post challenge. These results demonstrate that in a murine model, the MCSP DNA vaccine loses efficacy by 3 months. This is in contrast to the MCSP protein vaccine, which remain protective for more than five months, following only two doses of the vaccine (see Example 7, FIG. 4B). Therefore, the protein vaccine strategy disclosed herein, with fewer than three doses, elicits a surprisingly long lasting immune response, as compared to immunization with the equivalent DNA vaccine.

### Example 9. Antibody Response to Mouse or Human MIP-3α to Vaccination with the Combination of MCSP and Poly(I:C) Regimen

A potential issue for a highly immunogenic vaccine that includes a human product is whether a response is elicited to the human component of the vaccine, in this case the chemokine MIP-3α. Both human and mouse MIP-3α, which have 64% homology (Stowers, A. W., et al, Infect. Immun. 69:1536-46, 2001), will bind mouse CCR6 (Varona, R., et al., FEBS Lett. 440:188-94, 1998); human MIP-3α was used in the vaccine construct. To address this potential issue ELISA was used to determine whether specific antibody to either mouse or human MIP-3α could be detected in the serum of immunized mice. In the same assay, the same sera were evaluated for levels of anti-CSP antibody.

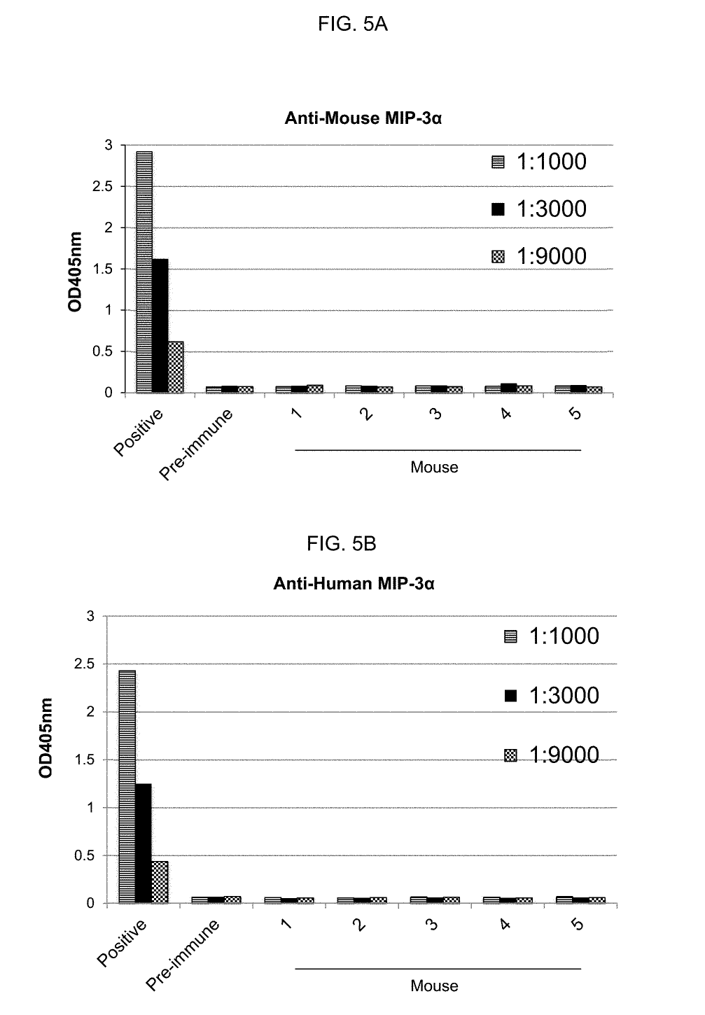

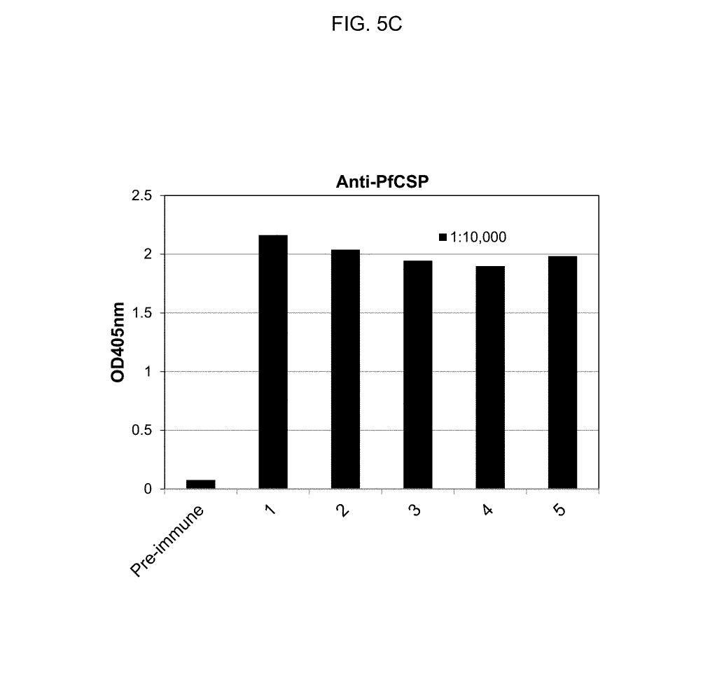

This analysis indicates that mice with high concentrations of anti-CSP antibody had no antibody that bound recombinant human or mouse MIP-3α after either one or two vaccinations. No anti-human MIP-3α nor anti-mouse MIP-3α antibodies could be detected either 3 or 23 weeks after the last immunization. FIG. 5 shows the absence of antibody response to the MIP-3α component of malaria vaccine. C57BL/6 mice were immunized with 20 μg hMCSP+Poly(I:C) twice at 3 week intervals.

Specific antibodies against human MIP-3α (A), mouse MIP-3α (B) or Pf CSP (C) were assayed by ELISA three weeks after the final immunization. Values shown represent absorbance at OD₄₀₅ nm from serum of pre-immune or immunized mice. No reactivity against either human or murine MIP-3α significantly exceeded that of the pre-immune sera (p=1.000).

### Example 10. Protection from Bloodstream Infection by hMfCSP Vaccine Plus Addavax™

The relative potency of poly(I:C) as an adjuvant was evaluated with two adjuvants that have been approved for this purpose by the FDA, alum and the squalene-based adjuvant used clinically as MF59® and in the research setting as Addavax™ (InvivoGen, San Diego, Calif.). The ability of these adjuvants to elicit and maintain an antibody response was compared.

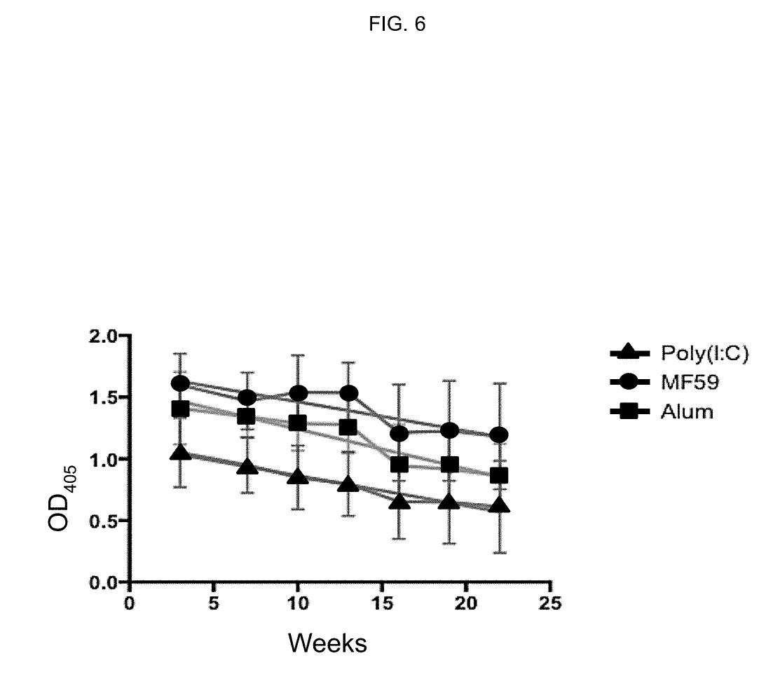

FIG. 6 shows the time course of antibody response with different adjuvants. Mice immunized twice with 20 μg MCSP and the indicated adjuvants (Poly(I:C), MF59® and Alum) at the recommended dosing were followed by ELISA for concentration of CSP-specific antibody (1:5000) dilution. P=0.36 difference in slopes; P<0.001 difference in magnitude of response. The slope of decline of specific antibody was equivalent with use of the different adjuvants, but Addavax™ elicited a significantly higher response, a difference that was sustained over the 23 weeks in which it was followed.

At 15 weeks after the final immunization, protection against 5,000 sporozoite intravenous challenge produced results similar to those observed using the poly(I:C) adjuvant. Additionally, five and 15 weeks after the last of two vaccinations, each of 26 immunized or control mice were challenged by exposure to eight transgenic P. berghei infected Anopheles stephensi mosquitoes obtained from the insectary of the Johns Hopkins Malaria Research Institute (Table 2).

TABLE 2
Protection from bloodstream infection by hMfCSP vaccine + Addavax.

| Time since | | Number with | P Value |
|---|---|---|---|
| last of two | | parasitemia/total | vs. Time |
| immunizations | Group | challenged | point control |
| 5 weeks | Immune (hMfCSP + | 1/7 | 0.008 |
| | Addavax) | | |
| | Non-immune | 6/7 | |
| 15 weeks | Immune (hMfCSP + | 2/6 | 0.01 |
| | Addavax) | | |
| | Non-immune | 6/6 | |

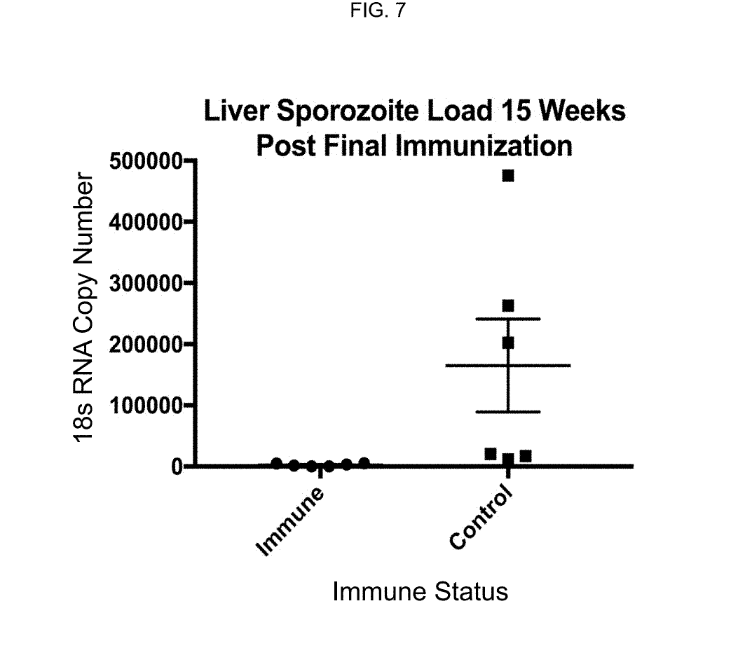

At the 15 week time point, highly significant protection against intravenous challenge with 5000 transgenic P. berghei was also observed, as measured by liver sporozoite infection (FIG. 7).

### Example 11. Vaccine Development in Non-Human Primates

This example is performed to examine the issue of whether a malaria vaccine platform that has elicited marked enhancement of antibody responses and extended protection compared to more traditional adjuvant plus vaccine approaches will also be more effective in eliciting responses from infant and juvenile non-human primates. As part of earlier studies with the DNA formulation of this vaccine platform, the inventors examined the response in two macaques to DNA priming followed by a single protein boost, all using the Vaxfectin adjuvant used in the DNA vaccine studies (described in U.S. Patent Publication US2015/0359869).

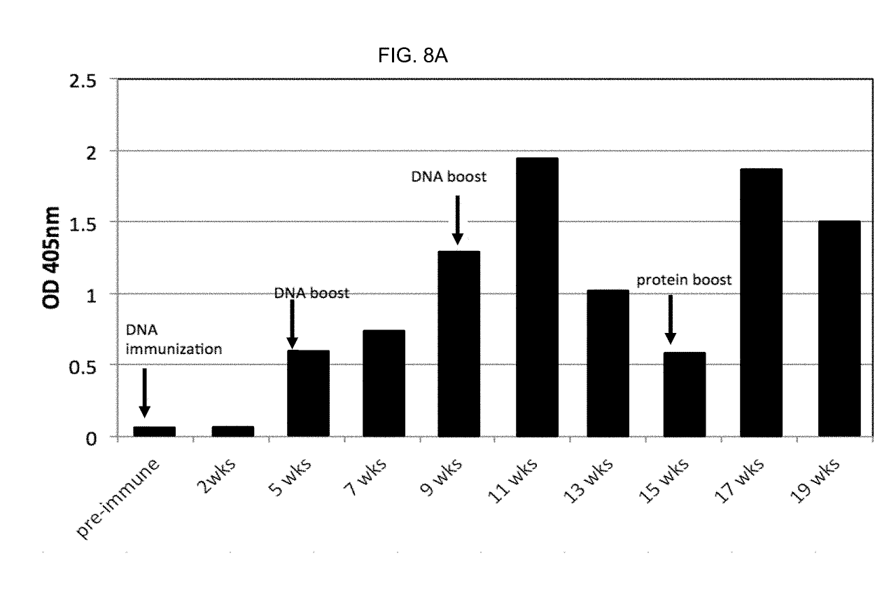

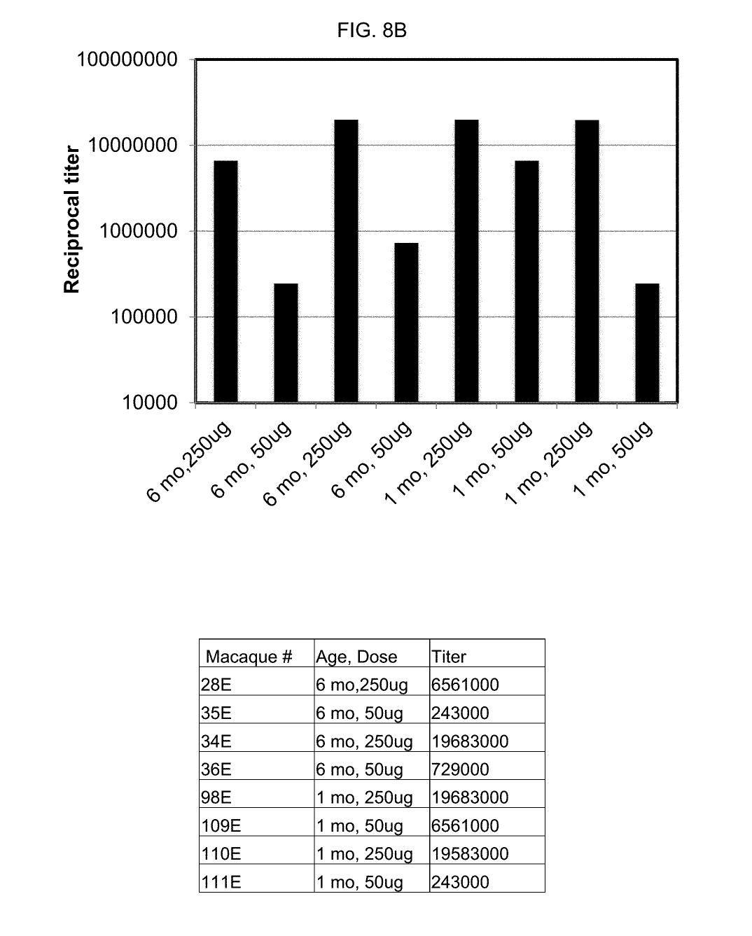

A representative result is shown in FIG. 8A, using a 1:5000 serum dilution. Results are presented to indicate the ability of macaques to respond to this vaccine construct as a DNA formulation with a protein boost, which provides support and rationale for performing comparable experiments in macaques using the protein vaccine disclosed herein.

Macaques are suitable for these studies because their immune ontogeny more closely parallels that of humans than does that of rodents or that of the New World monkeys that have been used as non-human primate models of malaria infection (Stowers, A. W., et al., Infect. Immun. 69:1536-46, 2001, Rosas J E, et al, Vaccine; 20:1707-10, 2002).

Although not susceptible to infection with either P. berghei or P. falciparum, macaques have been particularly useful for the study of immune responses in infant and juvenile animals for an array of different candidate vaccines, including malaria (Skinner, J. M., et al., Vaccine 29:8870-6, 2011; Rosario, M., et al., J. Virol. 84:7815-21, 2010; Walsh, D. S., et al., Am. J. Trop. Med. Hyg. 70:499-509, 2004; Polack, F. P., et al., Clinical and vaccine immunology: CVI 20:205-10, 2013; Pan, C. H., et al., Clinical and vaccine immunology: CVI 15:1214-21, 2008).

Most of these studies have simply evaluated the magnitude of antibody responses, although for some of the non-malaria studies, pathogen challenge was undertaken. In such cases, the protection results generally correlated well with the previous or subsequent human studies (Polack, F. P., et al., Clinical and vaccine immunology: CVI 20:205-10, 2013; Pan, C. H., et al., Clinical and vaccine immunology: CVI 15:1214-21, 2008).

To test the immunogenicity of the malaria protein vaccine disclosed herein, one month-old and six month-old macaques received either 50 μg or 250 μg of the MCSP vaccine in 125 μl of PBS formulated in an equivalent volume of the Addavax™ adjuvant, (Sorbitan trioleate (0.5% w/v) in squalene oil (5% v/v)—Tween 80 (0.5% w/v) in sodium citrate buffer (10 mM, pH 6.5), InvivoGen, San Diego, Calif.). The doses selected are based on previous experience in macaques and the published literature with candidate malaria protein vaccines in monkey model systems (Stowers, A. W., et al., Infect. Immun. 69:1536-46, 2001; Perlaza, B. L., et al., Eur. J. Immunol. 38:2610-5, 2008). At each age, there were two macaques per dosage group for a total of 8 macaques. The immunizations were repeated once three weeks after the first immunization, and sera were obtained three weeks later. FIG. 8B shows the antibody titer results, which are measured for specific antibody levels against Pf CSP NANP repeats. The endpoint titer is reported as the highest dilution of serum at which the absorbance was twice the value obtained using pre-immunization serum. These results show that the protein vaccine can elicit an antibody response in infant and very young non-human primates, the age groups in humans at greatest risk for severe or lethal malaria infection.

This immunization study is ongoing, and depending on the patterns of antibody response and decline, a third immunization may be initiated. Also, sera will be collected at two week intervals for nine months thereafter and frozen for in vitro and in vivo studies. Pre-immune sera from six month-old macaques will be used as control for all of the studies proposed below. If necessary, sera obtained at two week intervals may be pooled to obtain sufficient sera for the proposed challenge.

### Example 12. Examining In Vitro the Magnitude and Sustainability of Humoral Immune Responses

Sera obtained as described above will be quantitated for antibody concentration by limiting dilution analysis in a standard ELISA assay using 96 well plates coated with 5 μg of the 6-His-tagged segment of the PfCSP protein that includes 21 copies of the central NANP repeat region. The sera will also be titered for their ability to neutralize invasion of HepG2 cells using the standard transgenic sporozoite neutralization assay (Kumar, K. A., et al., J. Immunol. Methods 292:157-64, 2004). Briefly, 48 hours prior to addition of transgenic P. berghei sporozoites to the culture, HepG2-A16 cells (HB 8065; American Type Culture Collection) will be seeded at a density of 5×10⁴ cells/well in an 8-well chamber slide (Labtek).

Sera from immunized and nonimmune macaques, serially diluted three fold to 81-fold will be incubated with 2×10⁴ transgenic sporozoites isolated from the salivary glands of A. stephens mosquitoes and maintained on ice for 40 minutes prior to addition to the HepG2-A16 cells. After 72 hours of culture at 37° C., the cells will be harvested, RNA isolated and sporozoite copy number quantitated by qRT-PCR, using the HepG2 beta actin gene as a standardization control for input cells. The PfCSP-specific monoclonal antibody 2A10 will be used as a positive control in the assay.

Previous mouse studies by the inventors have provided guidance on what antibody titers are associated with protection and results from ELISA and neutralization assays, as well as results from the in vivo challenge studies described in Example 12, will be used to determine if booster immunizations will be required in the period during which the macaques are maintained.

### Example 13. Examining In Vivo the Ability of Passively Transferred Antibody Obtained from Immunized Macaques to Protect C57Bl/6 Mice Against Infection with Transgenic P. berghei Expressing P. falciparum CSP

The protective capability of sera from macaques receiving two immunizations was tested six weeks after the second immunization in both age groups. Sera from individual macaques was be evaluated in vitro, as above, and sera was pooled from each macaque pair within a group for these challenge studies. For in vivo studies mice receive intravenously 200 μl immune serum and were subsequently bled from the tail vein, 6-12 hours later, to evaluate whether ELISA titers observed in mouse sera approximate those observed with the macaque sera.

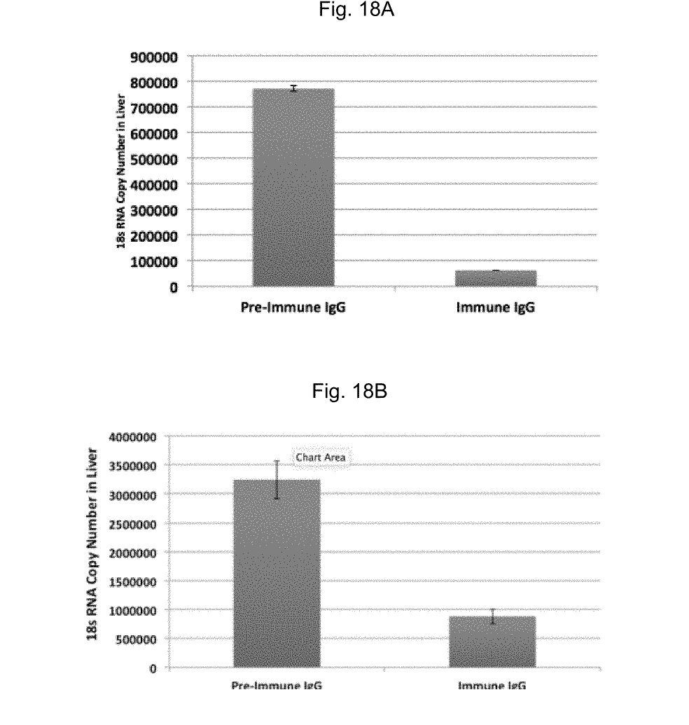

Murine recipients of antibody or serum from immunized and control macaques were challenged intravenously with 5,000 transgenic P. berghei, and 40 hours later the mice were euthanized, livers harvested and sporozoite 18s RNA copy number determined by qRT-PCR. Results showed that compared to preimmunized mice, immunized mice had a significant reduction in the transgenic P. berghei rRNA load in the liver with challenges after the second immunization (FIGS. 18A and 18B).

### Example 14. Protein Expression of vMIP-II, MC148, or MIP-3α Fused to Plasmodium CSP

The N-terminal 20 aa signaling sequence and the C-terminal 23 aa anchor region fused with Viral macrophage inflammatory protein 2 (vMIP2) or Molluscum contagiosum virus subtype 1 (MC148) was used to express vMIP2-CSP or MC148-CSP chimeric proteins. The sequences with c-myc tag in C-terminal were cloned into pET-47b fused with 6×His and proteins were expressed in BL21 DE3 competent cells. Expression of fusion proteins was confirmed by SDS-PAGE with Coomassie blue stain (FIG. 16).

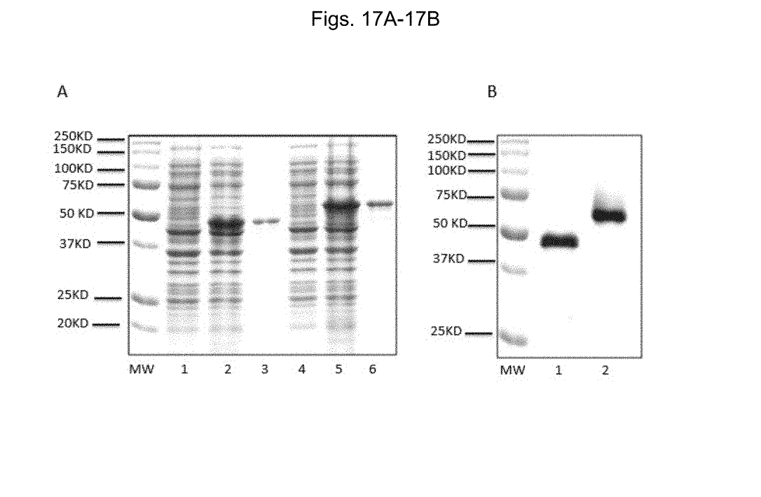

The codon-optimized P. falciparum CSP (3D7) DNA sequence containing deletions of the N-terminal 20 aa signaling sequence and the C-terminal 23 aa anchor region fused with human MIP-3α was used to express MIP-3α-CSP chimeric protein (MCSP). CSP and MCSP sequences with c-myc tag in C-terminal were cloned into pET-47b fused with 6×His and proteins were expressed in BL21 DE3 competent cells. Protein purification was undertaken using nickel-affinity chromatography. Expression and purification of MIP-3α-CSP fusion protein and the CSP protein was confirmed by SDS-PAGE with Coomassie blue stain and Western blot analysis using anti-CSP mAb (2A10) (FIGS. 17A and 17B).

### Example 15. Zika Virus-Based Vaccines

In addition to malaria-based vaccines, the disclosure provides new Zika virus vaccines consisting of Domain III of the E protein fused to each of three chemokine-like virus proteins. Recent studies using monoclonal antibodies have demonstrated the neutralizing and mouse-protective activity of antibodies targeting this region (Zhao H, et al. Cell 2016; 166:1016-27). Importantly these antibodies, which were elicited by immunization with Zika virus strains followed by boosting with recombinant protein, were not cross reactive with dengue viruses. Because of the structural similarities that exist between the E proteins of related flaviviruses, it is possible to draw on the experience of earlier studies with West Nile and dengue viruses, which indicated that, with proper preparation, recombinant protein vaccines could be constructed that elicited neutralizing responses and could be protective in both murine and non-human primate challenge models (Clements D E, et al. Vaccine 2010; 28:2705-15, Govindarajan D, et al. Vaccine 2015; 33:4105-16, Oliphant T, et al. J Virol 2007; 81:11828-39, McDonald W F, et al. J Infect Dis 2007; 195:1607-17).

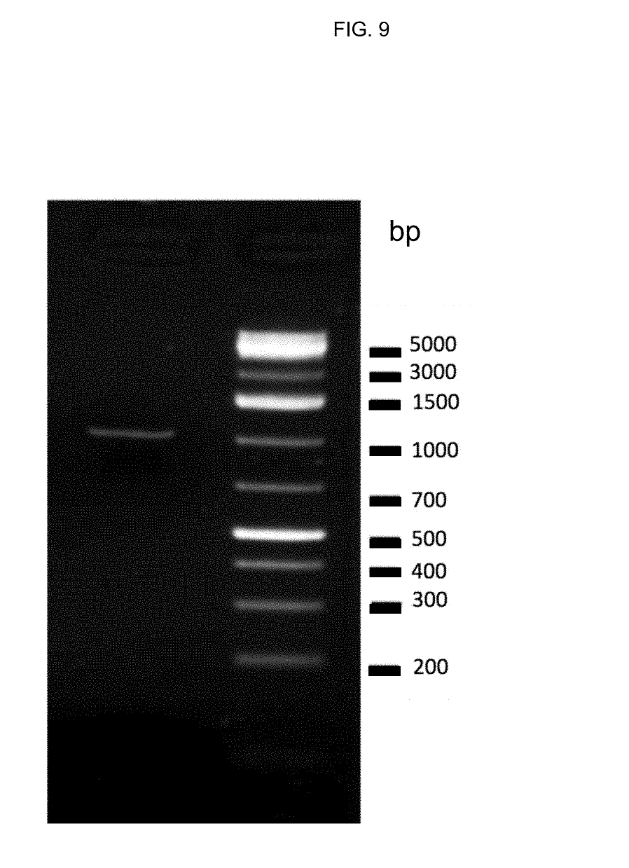

A disclosed vaccine construct employs the E protein Domain III region from Zika Fortaleza/2015 from Brazil. It differs by only a single amino acid (E→D, both negatively charged) from the Domain III region of Thailand/2014, SV0127/14, which was the source of initial RT-PCR of the E protein as shown FIG. 9. Because a vaccine constructed from this domain of West Nile virus and expressed from E. coli was protective in a West Nile virus challenge model (McDonald, W. F., et al. J. Infect. Dis. 195:1607-17, 2007) and because this region contains no N-glycosylation sites (Dubayle, J., et al., Vaccine 33:1360-8, 2015; Idris, F., et al., Arch. Virol. 161:1751-60, 2016), this vaccine construct can be readily produced in bacterial expression systems.

FIG. 9 demonstrates the reverse transcription and amplification of RNA encoding the viral E protein, truncated to remove the carboxyterminal membrane component. For generation of the viral EDIII component of the vaccine, RNA encoding the viral E protein from Fortaleza/2015 Zika virus, truncated to remove the carboxyterminal membrane component, is reverse transcribed and amplified. Primers (5′ 1171 TGGGGNAAYSRNTGYGGNYTNTTYGG 1 197 3′, SEQ ID NO: 15) and Unirev (5′ 2178 CCNCCHRNNGANCCRAARTCCCA 2155 3′, SEQ ID NO: 16) are used. This product is used as the template for amplification of the Domain III region, using Forward primer 5′-TCTAGAGCATTCACATTCACCAAGGTCC-3′ (SEQ ID NO: 17) and Reverse primer (with c-myc-tag): 5′-TTAATTAATCACAGATCCTCTTCTGAGATCAGTTTCTGTTCTTCTGCGGAACTCCTAT GCCAGTGGTG-3′ (SEQ ID NO: 18), which allows for cloning of the amplified product into the pET-47b (Novagen Inc., Madison, Wis.) expression vectors carrying the chemokines described below.

MIP-3α and virally-derived peptides that target the CCR8 receptor on both immature and mature DC, among other cells (Connolly S, Skrinjar M, Rosendahl A. Biochemical pharmacology 2012; 83:778-87) are used for the following experiments. The viral chemokine-like proteins include the molluscum contagiosum virus (MCV) chemokine MC148 (Luttichau HR, Stine J, Boesen T P, et al. J Exp Med 2000; 191:171-80), the human herpes virus 8-encoded vMIP-I (Dairaghi D J, et al. J Biol Chem 1999; 274:21569-74) and the Kaposi's sarcoma-associated herpesvirus chemokine vMIP-II (Kledal T N, et al. Science 1997; 277:1656-9). The ability of these viral chemokines to significantly enhance immune responses to a tumor-associated protein and to enhance mouse survival in that tumor model system in a manner analogous to MIP3α has been demonstrated (Biragyn A, et al. Blood 2002; 100:1153-9, Ruffini P A, et al. J Leukoc Biol 2004; 76:77-85). Cloning the Domain III DNA into these plasmid constructs is carried out using methods used for similar efforts with malaria vaccine constructs. Some of these viral chemokines actually antagonize signaling through their receptors (Luttichau HR, Stine J, Boesen T P, et al. J Exp Med 2000; 191:171-80), but this appears to have no effect on their ability to enhance immune responses (Ruffini P A, et al. J Leukoc Biol 2004; 76:77-85), supporting the concept that these chemokines act only as targeting molecules, with the adjuvant providing the signaling required to attract appropriate cells to the site of immunization and to initiate differentiation events associated with antigen presentation.

Unlike the situation with MIP3α, it is expected that antibody responses to the virally-derived chemokines would be elicited by the disclosed immunization protocol, which could potentially interfere with the ability of a given viral chemokine, on secondary immunization, to target vaccine antigen to the appropriate cells. For this reason, three different vaccine constructs will be developed, employing, if necessary, different viral chemokine constructs for subsequent rounds of booster immunizations.

The following experiments have been completed using the MIP-3α iDC targeting domain, and will be repeated using the viral chemokine iDC targeting domains, with similar results expected for each. Re-folding of expressed Zika antigen-MIP-3α chemokine fusion protein proceeded as described in Jaiswal, S. et al., Protein. Expr. Purif. 33:80-91 (2004) with protein purification undertaken using nickel-affinity chromatography employing the His tag incorporated in the vaccine platform (Qiagen, Valencia, Calif., USA). Endotoxin was removed by two-phase extraction with Triton X-114 (Chen, Z. et al., International Immunopharmacology 16:376-81, 2013).

Protein concentration was measured by Bradford assay (BioRad, Hercules, Calif.). Endotoxin concentration was determined using the ToxinSensor™ Chromogenic LAL Endotoxin Assay Kit (Genscript, Piscataway, N.J.). All protein used for immunization have final endotoxin levels below 10 EU/ml.

Purified proteins were separated by sodium dodecyl sulfate (SDS)-polyacrylamide gel electrophoresis (PAGE) for visualization with Coomassie Brilliant Blue staining and Western-blot analysis was undertaken using anti-myc antibody, also targeting a tag included in the construct. Secondary antibodies were alkaline phosphatase-conjugated anti-mouse antibodies (Jackson Immunoresearch, Inc., West Grove, Pa.), and staining was carried out with 5-bromo-4-chloro-3-indolylphosphate (BCIP) and nitro blue tetrazolium (NBT) solutions prepared from chemicals obtained from Sigma (St. Louis, Mo.). In a manner similar to that used for preparation of the vaccine protein, the Domain III protein minus the chemokine components was expressed and purified for use in ELISA assays and as a control vaccine.

As a measure of proper folding of the final vaccine construct, its ability to block Zika virus infection as described (Reddy P B, et al. Protein and peptide letters 2012; 19:509-19) in a Plaque Reduction Neutralization Test, described below, can be evaluated. In this study vaccine replaces antibody in the test. Should results suggest improper folding, alternative re-folding strategies can be employed. An alternative method for refolding the Zika protein employs the QuickFold Protein Refolding Kit (Athena Enzyme Systems, Baltimore, Md.) to screen additional buffer and refolding conditions to identify those that maintain the solubility of the vaccine; initial tests evaluate fifteen sets of conditions. Confirmation of correct refolding ultimately relies on neutralization assays after mouse immunization. An alternative to bacterial expression systems for yielding a soluble product after refolding is a eukaryotic expression system such as HEK293T cells or baculovirus expression systems. Products from these systems are expected to be less subject to refolding issues. Protection might also require targeting the larger E or prM-E proteins.

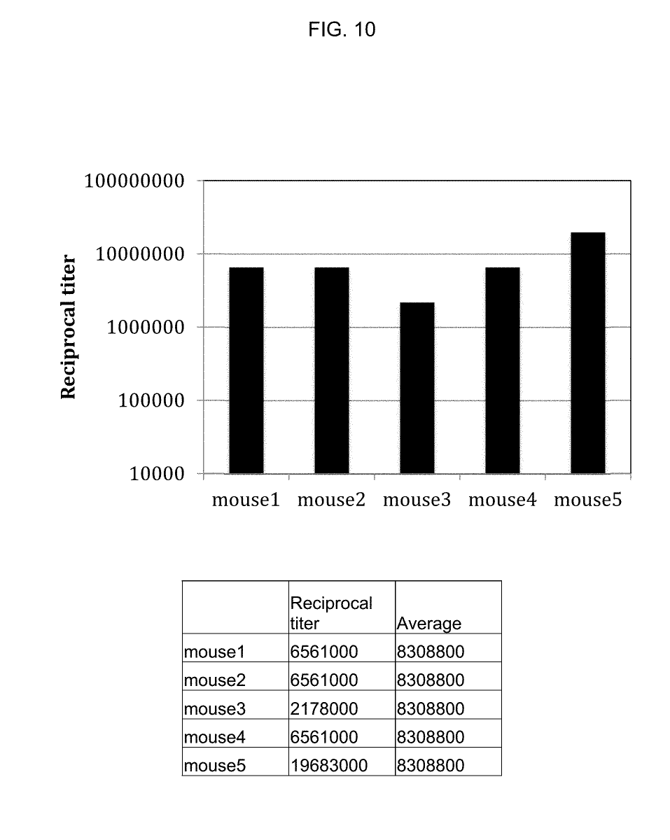

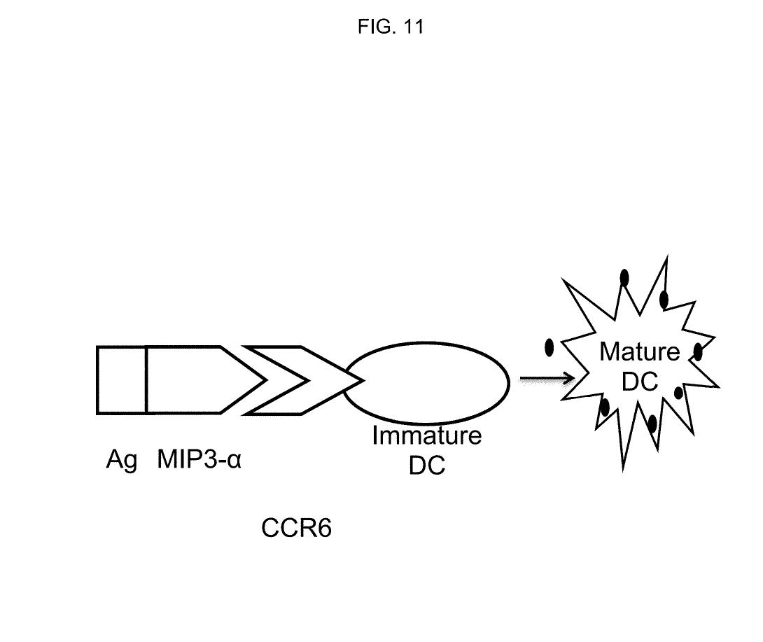

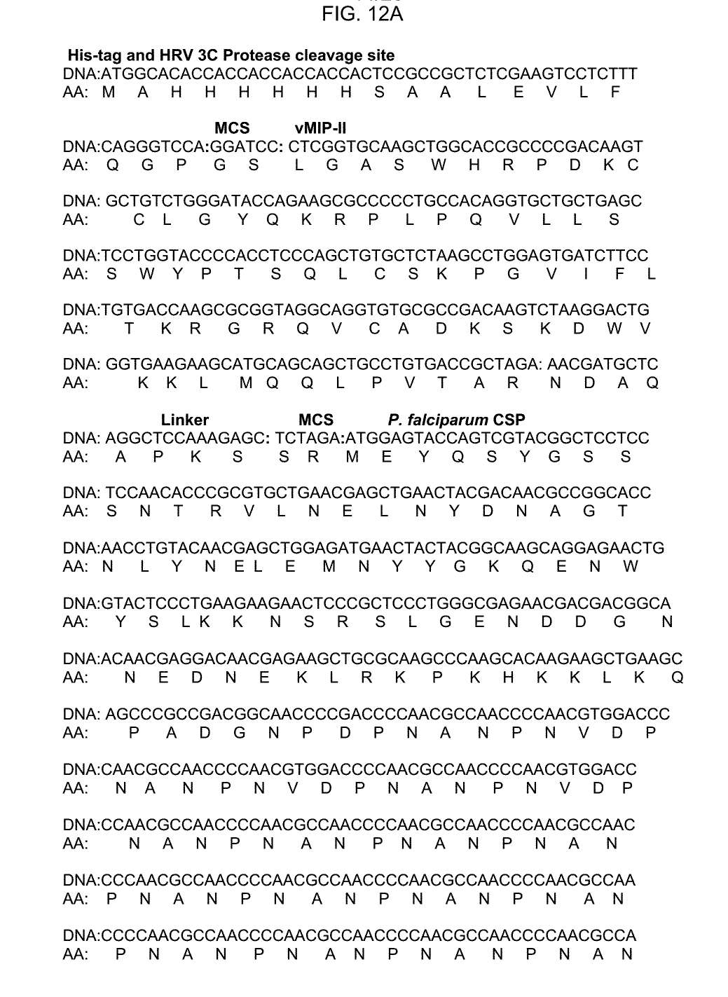

Initial testing of the immunogenicity of the MIP3α-Domain III vaccine has been conducted in C57Bl/6 mice, the background strain for the Irf3⁻, Irf5⁻, Irf7⁻ that will be used for subsequent challenge studies. Five 4-week old mice were each immunized with 20 μg of the Zika virus E protein EIII domain-MIP-3α vaccine construct plus 50 μg of the adjuvant MF59. Two immunizations separated by three weeks were administered and three weeks after the final immunization sera were obtained to determine antibody concentrations. Bleeds also preceded each of the immunizations, for use as pre-immunization controls. Specific antibody levels against Zika virus E protein EIII domain were determined (shown in FIG. 10). The endpoint titer is reported as the highest dilution of serum at which the absorbance was twice the value obtained using pre-immunization serum. These results indicate that the Zika-chemokine protein vaccine elicits a markedly elevated antibody response, beyond what would be expected, in a murine model.

Further experiments will be completed, including challenge with a range of vaccine doses. The vaccine construct containing EDIII not fused to a chemokine will also be employed to confirm the enhancement achieved by use of the chemokine fusion protein. Bleeds will be repeated at monthly intervals for six months. Neutralization titers will be determined using a recently described modification of standard PRNT assays (Abbink P, et al. Science 2016; 353:1129-32). Previous Zika vaccine protection studies in mice and monkeys have correlated protection with similarly performed ELISA titers at or in excess of 10³ with 50% neutralization titers in a similar range range (Abbink P, et al. Science 2016; 353:1129-32, Larocca R A, et al. Nature 2016; 536:474-8).

These experiments will be repeated with the Zika vaccines that include each of the viral chemokines, vMIP-1, vMIP-II and MC148, and these vaccines are also expected to elicit an antibody response against the Zika virus E protein EIII domain.

Based on previous malaria studies, attaining considerably higher titers than those reported by Abbink and Larocca (Abbink P, et al. Science 2016; 353:1129-32, Larocca R A, et al. Nature 2016; 536:474-8) is expected, but achievement of at least those levels by three weeks after a second immunization will determine if additional immunizations or increases in dosing will be undertaken before initating the long term studies. This decision will also be influenced by affinity measurements, as described below. It is appreciated that in vitro neutralization and ELISA results do not always correlate with in vivo protection studies, but these can serve as a surrogate for efficacy until the challenge studies are undertaken. These tests will be conducted following the WHO guidelines (Roehrig J T, et al. Viral immunology 2008; 21:123-32), as well as recently-described protocols (Van Hoeven N, et al. PloS one 2016; 11:e0149610), and will employ the Fortaleza/2015 Zika isolate.

Virus for these studies and in vivo challenge will be grown up in large batches on Vero cells and then aliquoted and frozen at −80° C. to establish a uniform stock of low passage virus. In addition to the standard Vero cell lines used in PRNT analyses, neutralization studies will be performed using Vero cells engineered to express the FcγR2α receptor. A number of investigators (Littaua R, et al. J Immunol 1990; 144:3183-6, Modhiran N, et al. PLoS neglected tropical diseases 2010; 4:e924) have shown that engagement of these receptors may contribute to antibody dependent enhancement of dengue virus infection. These cells will be used to address the impact of the concentration of E protein Domain III specific antibodies on Vero cell infection. In particular, antibody concentration and affinity dependency of enhancement will be examined as sera are obtained from immunized mice over time. A concentration effect can first be observed as antibody titers are reduced in the neutralization assay.

Experiments are performed to determine if there a difference between increased virus levels that result from simple dilution of neutralizing antibody on the Vero cell PRNT assay, vs. a further increase that might be observed during antibody dilution on the Vero-FcR cells. If enhancement increases as antibody concentration declines during titering in the PRNT assay then it would be important to define at what thresholds this effect might be observed.

Further, it would be important as well to know in what time frame after the final immunization that threshold is achieved in the immunized mice. Mice and humans may differ in antibody half-life, so antibody concentrations will be assayed over the six months that the C57Bl/6 mice are followed. Such studies might suggest whether and how often booster vaccinations might be required in high risk clinical settings. It is of relevance that there was a very slow decline of antibody concentrations with the vaccine construct when examined in the malaria mouse model system.

Affinity of antibodies for the Zika virus generated by the disclosed vaccines may be a critical component of whether those antibodies enhance or neutralize infection, as is the case for other flavivirus infections (Pierson T C, et al. Cell host & microbe 2007; 1:135-45, Klasse P J, Burton DR. Cell host & microbe 2007; 1:87-9, Sanchez M D, et al. Virology 2005; 336:70-82). Surface plasmon resonance (SPR) provides an important tool for studying protein-protein interactions. While monoclonal antibodies and protein antigens have traditionally been used for SPR analyses, there is ample evidence that average affinities for polyclonal antibodies can also be analyzed with this technology (Reddy S B, et al. BMC microbiology 2015; 15:133, Gutierrez-Aguirre I, et al. Analytical biochemistry 2014; 447:74-81, Khurana S, et al. PloS one 2014; 9:e95496). For this component, SPR affinity measurements are performed using antibody obtained by Protein G affinity purification of the antibody from immunized mice and purified Domain III protein for affinity measurements.

Results from this analysis will be correlated with results from protection vs. ADE analysis to identify an optimized combination of antibody concentration and affinity for subsequent clinical trials. Affinity would be expected to increase with increasing numbers of immunizations and results from these analyses should guide decisions on whether two, three or more immunizations would be required to achieve appropriate affinities, initially seeking to achieve K_{d}'s in the 10⁻⁵ to 10⁻⁴/sec⁻¹ range associated with neutralization in influenza model systems (Khurana S, et al. PloS one 2014; 9:e95496). Expectations will be adjusted based on determinations of correlations between affinity and neutralization using the Zika virus vaccines disclosed herein and Zika virus.

Mouse models for Zika infection have recently been described (Lazear H M, et al. Cell host & microbe 2016, Rossi S L, et al. Am J Trop Med Hyg 2016). These publications indicate that Irf3, 5 and 7 KO mice as old as eleven weeks can become symptomatic when challenged with Zika virus. These mice are deficient in critical components of the innate immune system but are capable of developing adaptive immune responses (Williams K L, et al. Ann N Y Acad Sci 2009; 1171 Suppl 1:E12-23, Partidos C D, et al. Vaccine 2011; 29:3067-73, Brewoo J N, et al. Vaccine 2012; 30:1513-20). Irf3⁻, Irf5⁻, Irf7⁻ triple KO mice can be used. The ability of vaccine constructs disclosed herein to elicit protective immunity to virus challenge will provide a foundation for rapid movement to non-human primate studies and clinical trials. For these studies, groups of six four-week old mice will initially receive the disclosed vaccine and the MF59 adjuvant. Control mice will receive PBS alone, PBS plus the MF59 adjuvant and EDIII without chemokine plus MF59. A second vaccine dose will be administered at six weeks of age. All vaccines and adjuvant will be administered intramuscularly. Dosage used will be based on the outcome of the studies in the C57Bl/6 mice. At eight weeks of age mice will be challenged with a dose of challenge virus previously determined to uniformly produce signs of infection and viremia in 100% of challenged mice, likely around 10⁵ PFU administered subcutaneously, as described [1]. Protection will be defined by absence of circulating virus on day 2 post challenge, failure to recover virus from spleen (both determined by qRT-PCR or the appearance of plaques on Vero cell cultures), absence of weight loss and absence of obvious neurologic deficits over a ten-day period, which will be monitored daily in the period after challenge.

To evaluate duration of protective immunity elicited by Zika vaccines disclosed herein, 200 μl of sera (chosen as the limit for safe i.v. injection) from C57Bl/6 mice obtained at different points after immunization with vaccine or control constructs up to six months post immunization will be administered via i.p. or tail vein route to groups of 4 week old triple KO mice. Two hours or two days post transfusion of immune serum (depending on transfusion route determined to be optimal), mice will be challenged as above and subsequently assayed as described for evidence of infection.

The significance of differences in continuous numerical results, e.g. antibody concentrations or qRT-PCR results, will be evaluated using a one-way analysis of variance. The significance of differences resulting from a dichotomous variable, e.g., development or absence of evidence of infection in challenged mice, will be determined using a Fisher exact test. A Pearson correlation analysis will be employed for examining correlations between antibody concentrations and affinities and neutralization. Stata or Prism software will be used for these analyses.

### Example 16. Challenge Studies with Triple Irf3, 5 and 7 KO Mice

Mouse models for Zika infection have been described (Lazear, H. M., et al., Cell Host & Microbe 19:720-30, 2016; Rossi, S. L., et al., Am. J. Trop. Med. Hyg. 94:1362-9, 2016). These publications indicate that mice as old as 11 weeks can become symptomatic when challenged with Zika virus. These mice are deficient in critical components of the innate immune system but are capable of developing adaptive immune responses (Williams, K. L., et al., Ann. N. Y. Acad. Sci. 1171 Suppl 1:E12-23, 2009; Partidos, C. D., et al., Vaccine 29:3067-73, 2011; Brewoo, J. N., et al., Vaccine 30:1513-20, 2012). Irf3⁻, Irf5⁻, Irf7⁻ triple KO mice can be used.

The ability of the disclosed vaccine constructs to elicit protective immunity to virus challenge will provide a foundation for rapid movement to non-human primate studies and clinical trials. For these studies, groups of six 4 week old mice will initially receive the vaccine and the MF59 adjuvant. Control mice will receive PBS alone or PBS plus the MF59 adjuvant. A second vaccine dose will be administered at 6 weeks of age. All vaccines and adjuvant will be administered intramuscularly. Dosage used will be based on the outcome of the studies in the C57Bl/6 mice.

At 7-8 weeks of age, mice will be challenged with a dose of the challenge virus previously determined to uniformly produce signs of infection and viremia in 100% of challenged mice, likely around 10⁵ PFU administered subcutaneously, as described (Rossi, S. L., et al., Am. J. Trop. Med. Hyg. 94:1362-9, 2016). Protection will be defined by absence of circulating virus on day 2 post challenge, failure to recover virus from spleen (both determined by qRT-PCR or the appearance of plaques on Vero cell cultures), absence of weight loss and absence of obvious neurologic deficits over a 10 day period, which will be monitored daily in the period after challenge.

To evaluate duration of protective immunity elicited by the disclosed vaccine, 200 μl of sera from C57Bl/6 mice obtained at different points after immunization with vaccine or control constructs up to six months post immunization will be administered via I.P. (intraperitoneal) route or tail vein to groups of 4 week old triple KO mice. Two hours or two days post transfusion of immune serum (depending on transfusion route determined to be optimal), mice will be challenged as above and subsequently assayed as described for evidence of infection.

The significance of differences in continuous numerical results, e.g. antibody concentrations or qRT-PCR results, will be evaluated using a one-way analysis of variance. The significance of differences resulting from a dichotomous variable, e.g., development or absence of evidence of infection in challenged mice, will be determined using a Fisher exact test. Stata or Prism software will be used for these analyses.

Based on previous experience with this vaccine construct, high levels of circulating antibody will likely be elicited. The protective effectiveness in the challenge model system will be tested. If protective responses are not achieved at first, the vaccine will be expressed in a eukaryotic expression system such as HEK293T cells and evaluated as described above. Protection might also require targeting the larger E or prM-E proteins. The initial work on development of a Domain III vaccine reflects the rapidity with which it is likely to be developed and expressed in high concentrations to address the current public health crisis.

As an alternative to the novel surface plasmon resonance system, standard ELISA based affinity measurements can be performed, which would be undertaken appreciating the issues related to proper folding of the target antigen.

### Example 17. Protein Expression of vMIP-II Fused to Zika Virus ED3

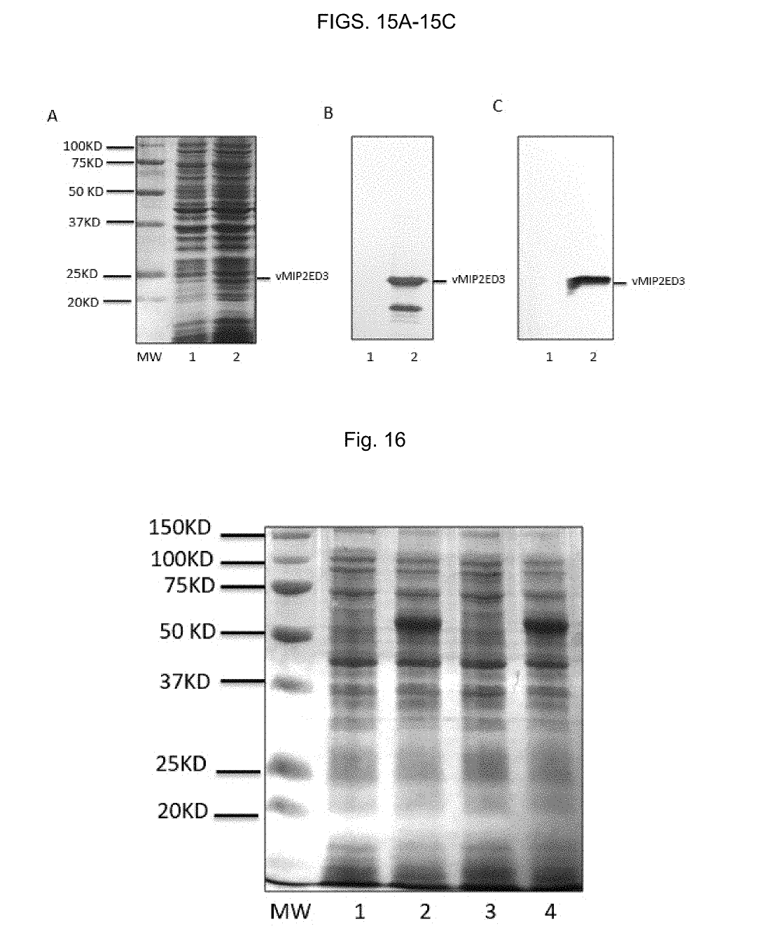

The codon-optimized Zika Virus (Brazil strain) envelope protein DIII domain (ED3) DNA sequence fused with Viral macrophage inflammatory protein 2 (vMIP2/vMIP-II) was used to express vMIP2-ED3 chimeric protein. vMIP2-ED3 sequence was cloned into pET-21a fused with 6×His in its C-terminal, and protein was expressed in BL21 DE3 competent cells. The expression was identified by SDS-PAGE with Coomassie blue stain and Western blot against anti-His and anti-ED3 antibodies (FIGS. 15A and 15B).

As will be understood by one of ordinary skill in the art, each embodiment disclosed herein can comprise, consist essentially of, or consist of its particular stated element, step, ingredient or component. Thus, the terms “include” or “including” should be interpreted to recite: “comprise, consist of, or consist essentially of.” The transition term “comprise” or “comprises” means includes and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically-significant reduction in a claimed vaccine or method's effectiveness in 1) reducing the risk of infection with the pathogen that is targeted by the vaccine, and/or 2) reducing the severity of infection caused by the pathogen that is targeted by the vaccine, both as measured by an animal model disclosed herein.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the”, and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Particular embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials is individually incorporated herein by reference in their entirety for their referenced teaching.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention can be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that can be employed are within the scope of the invention. Thus, by way of example alternative configurations of the present invention can be utilized in accordance with the teachings herein. Accordingly, the present invention is not limited to that precisely as shown and described.

What is claimed:

1. A vaccine, comprising (a) a fusion protein that comprises a Plasmodium circumsporozoite protein (CSP) linked to a chemokine that targets immature dendritic cells and (b) an adjuvant, wherein administration of the vaccine to subject elicits an immune response to Plasmodium CSP.

2. The vaccine of claim 1, wherein the chemokine is MIP-3α, MC148, vMIP-II, vMIP-I, or U83A.

3. The vaccine of claim 1, wherein the adjuvant comprises a squalene-based adjuvant.

4. The vaccine of claim 1, wherein the adjuvant comprises polyinosinic-polycytidylic acid (poly-IC) or a poly-IC derivative.

5. The vaccine of claim 1, wherein the vaccine is formulated at a dose of 50 μg-250 μg.

6. The vaccine of claim 1, wherein the immune response is an anti-CSP neutralizing antibody response.

7. The vaccine of claim 6, wherein the anti-CSP neutralizing antibody titer is detected in the subject following administration of fewer than three doses of the vaccine.

8. The vaccine of claim 7, wherein the anti-CSP neutralizing antibody titer is detected in the subject following administration of two doses of the vaccine.

9. The vaccine of claim 6, wherein the anti-CSP neutralizing antibody titer is at least 10-fold greater than a control, wherein the control is an anti-CSP neutralizing antibody titer elicited in a subject administered a DNA vaccine comprising an adjuvant and a nucleic acid encoding a fusion protein that comprises a Plasmodium CSP linked to a chemokine that targets immature dendritic cells.

10. The vaccine of claim 6, wherein a reciprocal anti-CSP neutralizing antibody titer of at least 10⁵ is detected in the subject at 3 weeks following administration of fewer than three doses of the vaccine.

11. The vaccine of claim 10, wherein the reciprocal anti-CSP neutralizing antibody titer of at least 10⁵ is detected in the subject at 3 weeks following administration of no more than two doses of the vaccine.

12. The vaccine of claim 1, wherein following administration of the vaccine to a subject, the number of inflammatory cells attracted to the site of vaccine administration is at least 50% greater compared to a control, wherein the control is the number of inflammatory cells attracted to the site of vaccine administration in a subject administered an adjuvant-free vaccine comprising a fusion protein that comprises a Plasmodium CSP linked to a chemokine that targets immature dendritic cells.

13. The vaccine of claim 1, wherein following administration of the vaccine to a subject, the Plasmodium parasitic load is reduced by at least 90% in the subject after challenge with Plasmodium, relative to the Plasmodium parasitic load detected in an unvaccinated control subject after challenge with Plasmodium.

14. The vaccine of claim 1, wherein the Plasmodium CSP comprises the sequence identified by SEQ ID NO: 26.

15. The vaccine of claim 2, wherein the fusion protein comprises the sequence identified by any one of SEQ ID NO: 38, SEQ ID NO: 39, or SEQ ID NO: 40.

16. A method, comprising administering to a subject the vaccine of claim 1, wherein the vaccine elicits an immune response to Plasmodium CSP.

17. The method of claim 16, wherein the vaccine is administered intramuscularly.

18. The method of claim 14, wherein the method comprises administering a prime dose of the vaccine and a boost dose of the vaccine.

19. The method of claim 18, wherein the method comprises administering no more than a prime dose of the vaccine and a boost dose of the vaccine.

20. The method of claim 14, wherein the subject is a child under the age of 5 years.

21. The method of claim 20, wherein the subject is an infant under the age of 1 year.

22. A vaccine, comprising (a) a fusion protein that comprises a Plasmodium circumsporozoite protein (CSP) linked to MIP-3α and (b) a squalene-based adjuvant, wherein administration of the vaccine to subject elicits an immune response to Plasmodium CSP.

23. A method, comprising intramuscularly administering to a subject no more than two doses of the vaccine of claim 22, wherein a reciprocal anti-CSP neutralizing antibody titer of at least 10⁵ is detected in the subject following the second dose of the vaccine.

24. A vaccine, comprising (a) a fusion protein that comprises a Zika virus antigen linked to a chemokine that targets immature dendritic cells and (b) an adjuvant, wherein administration of the vaccine to subject elicits an immune response to Zika virus antigen.

25. A method, comprising administering to a subject the vaccine of claim 24, wherein the vaccine elicits an immune response to Zika virus.


SEQUENCE LISTING (42 sequences)

SEQ ID NO: 1 - protein, 14 aa, Artificial Sequence
  Synthetic polypeptide
EFNDAQAPKSGSSR

SEQ ID NO: 2 - DNA, 26 bp, Artificial Sequence
  Synthetic polynucleotide
  misc_feature (6)..(6): n is a, c, g, or t
  misc_feature (12)..(12): n is a, c, g, or t
  misc_feature (18)..(18): n is a, c, g, or t
  misc_feature (21)..(21): n is a, c, g, or t
tggggnaaysrntgyggnytnttygg

SEQ ID NO: 3 - DNA, 23 bp, Artificial Sequence
  Synthetic polynucleotide
  misc_feature (3)..(3): n is a, c, g, or t
  misc_feature (8)..(9): n is a, c, g, or t
  misc_feature (12)..(12): n is a, c, g, or t
ccncchrnnganccraartccca

SEQ ID NO: 4 - protein, 70 aa, Artificial Sequence
  Synthetic polypeptide
LGASWHRPDKCCLGYQKRPLPQVLLSSWYPTSQLCSKPGVIFLTKRGRQVCADKSKDWVKKLMQQLPVTA

SEQ ID NO: 5 - protein, 94 aa, Artificial Sequence
  Synthetic polypeptide
MDTKGILLVAVLTALLCLQSGDTLGASWHRPDKCCLGYQKRPLPQVLLSSWYPTSQLCSKPGVIFLTKRGRQVCADKSKDWVKKLMQQLPVTAR

SEQ ID NO: 6 - DNA, 20 bp, Artificial Sequence
  Synthetic polynucleotide
gtccctcaccctcccaaaag

SEQ ID NO: 7 - protein, 8 aa, Artificial Sequence
  Synthetic polypeptide
NDAQAPKS

SEQ ID NO: 8 - protein, 19 aa, Artificial Sequence
  Synthetic polypeptide
MAHHHHHHSAALEVLFQGP

SEQ ID NO: 9 - protein, 13 aa, Artificial Sequence
  Synthetic polypeptide
SAEEQKLISEEDL

SEQ ID NO: 10 - protein, 424 aa, Artificial Sequence
  Synthetic polypeptide
MAHHHHHHSAALEVLFQGPGYQDPNSVQALARPTSAASNFDCCLGYTDRILHPKFIVGFTRQLANEGCDINAIIFHTKKKLSVCANPKQTWVKYIVRLLSKKVKNMEFNDAQAPKSGSSRMEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCRSAEEQKLISEEDL

SEQ ID NO: 11 - DNA, 26 bp, Artificial Sequence
  Synthetic polynucleotide
tgggagattggttttgacgtttatgt

SEQ ID NO: 12 - DNA, 30 bp, Artificial Sequence
  Synthetic polynucleotide
aagcattaaataaagcgaatacatccttac

SEQ ID NO: 13 - DNA, 20 bp, Artificial Sequence
  Synthetic polynucleotide
gtccctcaccctcccaaaag

SEQ ID NO: 14 - DNA, 21 bp, Artificial Sequence
  Synthetic polynucleotide
gctgcctcaacacctcaaccc

SEQ ID NO: 15 - DNA, 26 bp, Artificial Sequence
  Synthetic polynucleotide
  misc_feature (6)..(6): n is a, c, g, or t
  misc_feature (12)..(12): n is a, c, g, or t
  misc_feature (18)..(18): n is a, c, g, or t
  misc_feature (21)..(21): n is a, c, g, or t
tggggnaaysrntgyggnytnttygg

SEQ ID NO: 16 - DNA, 23 bp, Artificial Sequence
  Synthetic polynucleotide
  misc_feature (3)..(3): n is a, c, g, or t
  misc_feature (8)..(9): n is a, c, g, or t
  misc_feature (12)..(12): n is a, c, g, or t
ccncchrnnganccraartccca

SEQ ID NO: 17 - DNA, 28 bp, Artificial Sequence
  Synthetic polynucleotide
tctagagcattcacattcaccaaggtcc

SEQ ID NO: 18 - DNA, 68 bp, Artificial Sequence
  Synthetic polynucleotide
ttaattaatcacagatcctcttctgagatcagtttctgttcttctgcggaactcctatgccagtggtg

SEQ ID NO: 19 - protein, 406 aa, Artificial Sequence
  Synthetic polypeptide
MAHHHHHHSAALEVLFQGPGSLGASWHRPDKCCLGYQKRPLPQVLLSSWYPTSQLCSKPGVIFLTKRGRQVCADKSKDWVKKLMQQLPVTARNDAQAPKSSRMEYQSYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCRSAEEQKLISEEDL

SEQ ID NO: 20 - protein, 415 aa, Artificial Sequence
  Synthetic polypeptide
MAHHHHHHSAALEVLFQGPGSLSRRKCCLNPTNRPIPRPLLQDLDKVDYQPMGHDCGREAFRVTLQDGRQGCVSVGNQSLLDWLKGHKDLCPRMWPGCESLNDAQAPKSSRMEYQSYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCRSAEEQKLISEEDL

SEQ ID NO: 21 - protein, 206 aa, Artificial Sequence
  Synthetic polypeptide
MAHHHHHHSAALEVLFQGPGSLGASWHRPDKCCLGYQKRPLPQVLLSSWYPTSQLCSKPGVIFLTKRGRQVCADKSKDWVKKLMQQLPVTARNDAQAPKSRSAFTFTKIPAETLHGTVTVEVQYAGTDGPCKVPAQMAVDMQTLTPVGRLITANPVITEGTENSKMMLELDPPFGDSYIVIGVGEKKITHHWHRSEEQKLISEEDL

SEQ ID NO: 22 - protein, 215 aa, Artificial Sequence
  Synthetic polypeptide
MAHHHHHHSAALEVLFQGPGSLSRRKCCLNPTNRPIPRPLLQDLDKVDYQPMGHDCGREAFRVTLQDGRQGCVSVGNQSLLDWLKGHKDLCPRMWPGCESLNDAQAPKSRSAFTFTKIPAETLHGTVTVEVQYAGTDGPCKVPAQMAVDMQTLTPVGRLITANPVITEGTENSKMMLELDPPFGDSYIVIGVGEKKITHHWHRSEEQKLISEEDL

SEQ ID NO: 23 - protein, 212 aa, Artificial Sequence
  Synthetic polypeptide
MAHHHHHHSAALEVLFQGPGYQAASNFDCCLGYTDRILHPKFIVGFTRQLANEGCDINAIIFHTKKKLSVCANPKQTWVKYIVRLLSKKVKNMEFNDAQAPKSGSPTSAFTFTKIPAETLHGTVTVEVQYAGTDGPCKVPAQMAVDMQTLTPVGRLITANPVITEGTENSKMMLELDPPFGDSYIVIGVGEKKITHHWHRSEEQKLISEEDL

SEQ ID NO: 24 - protein, 13 aa, Artificial Sequence
  Synthetic polypeptide
NDAQAPKRSTGTS

SEQ ID NO: 25 - protein, 12 aa, Artificial Sequence
  Synthetic polypeptide
NDAQAPKRSTKL

SEQ ID NO: 26 - protein, 291 aa, Plasmodium
MEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCR

SEQ ID NO: 27 - DNA, 621 bp, Artificial Sequence
  Synthetic polynucleotide
atggcacatcaccaccaccatcactccgcggctcttgaagtcctctttcagggacccggatccctgggagcgtcctggcatagaccggacaagtgctgtctcggttaccagaaaagaccattaccacaggtgcttctgtccagctggtaccccacctcccaactgtgcagcaagccgggtgtgatatttttgacaaagcgtggtcgccaggtgtgtgccgacaaatcgaaagactgggtgaagaagctgatgcagcaattaccagtcactgctcgcaacgacgctcaggcgccgaagagtagatctgcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaggcactgagaactctaagatgatgctggaacttgatccaccatttggggactcttacattgtcataggagtcggggagaagaagatcacccaccactggcacaggagtgaagaacagaaactgatctcagaagaggatctgtga

SEQ ID NO: 28 - DNA, 648 bp, Artificial Sequence
  Synthetic polynucleotide
atggcacatcaccaccaccatcactccgcggctcttgaagtcctctttcagggacccggatccctctcgagacggaaatgttgtttgaatcctacaaatcgtccgataccgaggcctttactgcaggatctagacaaagtcgattatcagccgatgggacatgactgcggacgggaagctttcagagtgacgctgcaagacggaagacaaggctgtgttagcgttggtaaccagagtttactagactggctgaagggacacaaggatctctgcccgcggatgtggcccgggtgcgagtctctgaacgacgctcaggcgccgaagagtagatctgcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaggcactgagaactctaagatgatgctggaacttgatccaccatttggggactcttacattgtcataggagtcggggagaagaagatcacccaccactggcacaggagtgaagaacagaaactgatctcagaagaggatctgtga

SEQ ID NO: 29 - DNA, 1221 bp, Artificial Sequence
  Synthetic polynucleotide
atggcacaccaccaccaccaccactccgccgctctcgaagtcctctttcagggtccaggatccctcggtgcaagctggcaccgccccgacaagtgctgtctgggataccagaagcgccccctgccacaggtgctgctgagctcctggtaccccacctcccagctgtgctctaagcctggagtgatcttcctgtgaccaagcgcggtaggcaggtgtgcgccgacaagtctaaggactgggtgaagaagcatgcagcagctgcctgtgaccgctagaaacgatgctcaggctccaaagagctctagaatggagtaccagtcgtacggctcctcctccaacacccgcgtgctgaacgagctgaactacgacaacgccggcaccaacctgtacaacgagctggagatgaactactacggcaagcaggagaactggtactccctgaagaagaactcccgctccctgggcgagaacgacgacggcaacaacgaggacaacgagaagctgcgcaagcccaagcacaagaagctgaagcagcccgccgacggcaaccccgaccccaacgccaaccccaacgtggaccccaacgccaaccccaacgtggaccccaacgccaaccccaacgtggaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgtggaccccaacgccaaccccaacgccaaccccaacaagaacaaccagggcaacggccagggccacaacatgcccaacgaccccaaccgcaacgtggacgagaacgccaacgccaactccgccgtgaagaacaacaacaacgaggagccctccgacaagcacatcaaggagtacctgaacaagatccagaactccctgtccaccgagtggtccccctgctccgtgacctgcggcaacggcatccaggtgcgcatcaagcccggctccgccaacaagcccaaggacgagctggactacgccaacgacatcgagaagaaaatctgcaagatggagaagtgcagatccgcagaagaacagaaactgatctcagaagaggatctgtga

SEQ ID NO: 30 - DNA, 1248 bp, Artificial Sequence
  Synthetic polynucleotide
atggcacaccaccaccaccaccactccgccgctctcgaagtcctctttcagggtccaggatccctcagtcgtagaaaatgttgtctgaaccctaccaaccgcccaatccccaggcctctgctccaggatctggacaaggtggactaccagcccatgggccacgactgcggaagagaggccttccgtgtgaccctccaggacggtagacagggctgcgtgtctgtgggtaaccagagcctgctggactggctgaagggccacaaggacctgtgccctagaatgtggcctggttgtgaaagcctgaacgatgctcaggctccaaagagctctagaatggagtaccagtcgtacggctcctcctccaacacccgcgtgctgaacgagctgaactacgacaacgccggcaccaacctgtacaacgagctggagatgaactactacggcaagcaggagaactggtactccctgaagaagaactcccgctccctgggcgagaacgacgacggcaacaacgaggacaacgagaagctgcgcaagcccaagcacaagaagctgaagcagcccgccgacggcaaccccgaccccaacgccaaccccaacgtggaccccaacgccaaccccaacgtggaccccaacgccaaccccaacgtggaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgtggaccccaacgccaaccccaacgccaaccccaacaagaacaaccagggcaacggccagggccacaacatgcccaacgaccccaaccgcaacgtggacgagaacgccaacgccaactccgccgtgaagaacaacaacaacgaggagccctccgacaagcacatcaaggagtacctgaacaagatccagaactccctgtccaccgagtggtccccctgctccgtgacctgcggcaacggcatccaggtgcgcatcaagcccggctccgccaacaagcccaaggacgagctggactacgccaacgacatcgagaagaaaatctgcaagatggagaagtgcagatccgcagaagaacagaaactgatctcagaagaggatctgtga

SEQ ID NO: 31 - DNA, 1275 bp, Artificial Sequence
  Synthetic polynucleotide
atggcacatcaccaccaccatcactccgcggctcttgaagtcctctttcagggacccgggtaccaggatccgaattctgtacaggccttggcgcgcccgacgtccgcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtggatcctctagaatggagtaccagtcgtacggctcctcctccaacacccgcgtgctgaacgagctgaactacgacaacgccggcaccaacctgtacaacgagctggagatgaactactacggcaagcaggagaactggtactccctgaagaagaactcccgctccctgggcgagaacgacgacggcaacaacgaggacaacgagaagctgcgcaagcccaagcacaagaagctgaagcagcccgccgacggcaaccccgaccccaacgccaaccccaacgtggaccccaacgccaaccccaacgtggaccccaacgccaaccccaacgtggaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgccaaccccaacgtggaccccaacgccaaccccaacgccaaccccaacaagaacaaccagggcaacggccagggccacaacatgcccaacgaccccaaccgcaacgtggacgagaacgccaacgccaactccgccgtgaagaacaacaacaacgaggagccctccgacaagcacatcaaggagtacctgaacaagatccagaactccctgtccaccgagtggtccccctgctccgtgacctgcggcaacggcatccaggtgcgcatcaagcccggctccgccaacaagcccaaggacgagctggactacgccaacgacatcgagaagaaaatctgcaagatggagaagtgcagatccgcagaagaacagaaactgatctcagaagaggatctgtga

SEQ ID NO: 32 - DNA, 639 bp, Artificial Sequence
  Synthetic polynucleotide
atggcacatcaccaccaccatcactccgcggctcttgaagtcctctttcagggacccgggtaccaggcagcaagcaactttgactgctgtcttggatacacagaccgtattcttcatcctaaatttattgtgggcttcacacggcagctggccaatgaaggctgtgacatcaatgctatcatctttcacacaaagaaaaagttgtctgtgtgcgcaaatccaaaacagacttgggtgaaatatattgtgcgtctcctcagtaaaaaagtcaagaacatggaattcaacgacgctcaggcgccgaagagtggatccccgacgtccgcgttcacattcaccaagatcccggctgaaacactgcacgggacagtcacagtggaggtacagtacgcagggacagatggaccttgcaaggttccagctcagatggcggtggacatgcaaactctgaccccagttgggaggttgataaccgctaaccccgtaatcactgaaggcactgagaactctaagatgatgctggaacttgatccaccatttggggactcttacattgtcataggagtcggggagaagaagatcacccaccactggcacaggagtgaagaacagaaactgatctcagaagaggatctgtga

SEQ ID NO: 33 - protein, 10 aa, Artificial Sequence
  Synthetic polypeptide
NDAQAPKSGS

SEQ ID NO: 34 - protein, 73 aa, Artificial Sequence
  Synthetic polypeptide
AASNFDCCLGYTDRILHPKFIVGFTRQLANEGCDINAIIFHTKKKLSVCANPKQTWVKYIVRLLSKKVKNMEF

SEQ ID NO: 35 - protein, 70 aa, Artificial Sequence
  Synthetic polypeptide
GASWHRPDKCCLGYQKRPLPQVLLSSWYPTSQLCSKPGVIFLTKRGRQVCADKSKDWVKKLMQQLPVTAR

SEQ ID NO: 36 - protein, 80 aa, Artificial Sequence
  Synthetic polypeptide
LSRRKCCLNPTNRPIPRPLLQDLDKVDYQPMGHDCGREAFRVTLQDGRQGCVSVGNQSLLDWLKGHKDLCPRMWPGCESL

SEQ ID NO: 37 - protein, 101 aa, Zika Virus
VSYSLCTAAFTFTKIPAETLHGTVTVEVQYAGTDGPCKVPAQMAVDMQTLTPVGRLITANPVITEGTENSKMMLELDPPFGDSYIVIGVGEKKITHHWHRS

SEQ ID NO: 38 - protein, 376 aa, Artificial Sequence
  Synthetic polypeptide
AASNFDCCLGYTDRILHPKFIVGFTRQLANEGCDINAIIFHTKKKLSVCANPKQTWVKYIVRLLSKKVKNMEFNDAQAPKSGSSRMEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCR

SEQ ID NO: 39 - protein, 374 aa, Artificial Sequence
  Synthetic polypeptide
GASWHRPDKCCLGYQKRPLPQVLLSSWYPTSQLCSKPGVIFLTKRGRQVCADKSKDWVKKLMQQLPVTARNDAQAPKRSTGTSMEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCR

SEQ ID NO: 40 - protein, 384 aa, Artificial Sequence
  Synthetic polypeptide
LSRRKCCLNPTNRPIPRPLLQDLDKVDYQPMGHDCGREAFRVTLQDGRQGCVSVGNQSLLDWLKGHKDLCPRMWPGCESLNDAQAPKRSTGTSMEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCR

SEQ ID NO: 41 - protein, 183 aa, Artificial Sequence
  Synthetic polypeptide
GASWHRPDKCCLGYQKRPLPQVLLSSWYPTSQLCSKPGVIFLTKRGRQVCADKSKDWVKKLMQQLPVTARNDAQAPKRSTKLVSYSLCTAAFTFTKIPAETLHGTVTVEVQYAGTDGPCKVPAQMAVDMQTLTPVGRLITANPVITEGTENSKMMLELDPPFGDSYIVIGVGEKKITHHWHRS

SEQ ID NO: 42 - protein, 193 aa, Artificial Sequence
  Synthetic polypeptide
LSRRKCCLNPTNRPIPRPLLQDLDKVDYQPMGHDCGREAFRVTLQDGRQGCVSVGNQSLLDWLKGHKDLCPRMWPGCESLNDAQAPKRSTKLVSYSLCTAAFTFTKIPAETLHGTVTVEVQYAGTDGPCKVPAQMAVDMQTLTPVGRLITANPVITEGTENSKMMLELDPPFGDSYIVIGVGEKKITHHWHRS